(19) United States
(12) Patent Application Publication
(10) Pub. No.: US 2022/0296495 A1
(43) Pub. Date: Sep. 22, 2022

(54) DEODORANT COMPOSITIONS

(71) Applicants: Marisa Ann PLESCIA, Eagan, MN (US); Robert A. GANZ, Minnetonka, MN (US); Mo E. SAREMI, Eagan, MN (US); G&S Laboratories, Inc., Eagan, MN (US)

(72) Inventors: Marisa Ann Plescia, Eagan, MN (US); Robert A. Ganz, Minnetonka, MN (US); Mo E. Saremi, Eagan, MN (US)

(21) Appl. No.: 17/618,256

(22) PCT Filed: Jun. 11, 2020
(86) PCT No.: PCT/US2020/037299

§ 371 Date: Dec. 10, 2021

Related U.S. Application Data
(60) Provisional application No. 62/860,157, filed on Jun. 11, 2019.
(60) Provisional application No. 62/923,267, filed on Oct. 18, 2019.
(60) Provisional application No. 62/958,622, filed on Jan. 8, 2020.

(51) Int. Cl. A61K 8/9728; A61K 8/34; A61K 8/49; A61K 8/60; A61K 8/37; A61K 8/368; A61K 8/9789; A61K 8/92; A61K 8/9794; A61Q 15/00

(52) U.S. Cl. CPC A61K 8/9728; A61K 8/342; A61K 8/4953; A61K 8/604; A61K 8/375; A61K 8/4926; A61K 8/368; A61K 8/9789; A61K 8/927; A61K 8/922; A61K 8/9794; A61Q 15/00

(57) ABSTRACT

This disclosure relates to topical personal care compositions comprising one or more of a yeast component; a Bacillaceae extract; and 13-methyltetradecanoate; and B) one or more of: a phytochemical; an organic acid; and an antimicrobial peptide. In some embodiments, a composition as described herein can include a yeast component present in an amount of at least about 0.5% w/w of the composition; caffeine present in an amount of about 0.05% to about 5% w/w of the composition; a lemon grass extract present in an amount of at least about 0.01% to about 5% w/w of the composition; and an citrus fruit extract present in an amount of at least about 0.01% to about 5% w/w of the composition. In some embodiments, the composition further comprises a dermatologically acceptable carrier. Such compositions are useful for reducing body odor.

### CROSS-REFERENCE TO RELATED APPLICATIONS

This application claims priority to U.S. Provisional Application No. 62/958,622 filed on Jan. 8, 2020, U.S. Provisional Application No. 62/923,267 filed on Oct. 18, 2019, and U.S. Provisional Application No. 62/860,157 filed on Jun. 11, 2019, each of which is herein incorporated by reference in its entirety.

### TECHNICAL FIELD

The present disclosure relates to topical personal care compositions comprising one or more of a yeast component, a Bacillaceae extract, a phytochemical, an organic acid, and an antimicrobial peptide. Such compositions are useful for reducing body odor.

### BACKGROUND

The everyday utilization of personal care products, such as deodorants and/or antiperspirants, are common among consumers, with an estimated 2018 global market of 72.7 billion U.S. dollars (see “Size of the Global Antiperspirant and Deodorant Market 2012-2024 Statistic.” Statista). These products seek to help with body malodor and/or excessive sweating, brought on both by the effects of the body's biochemistry and of daily activities, such as exercise.

An average human has approximately 2.5 million sweat glands on the surface of the skin (see Bensouilah and Buck. Aromadermatology: Aromatherapy in the Treatment and Care of Common Skin Conditions. Radcliffe Publishing, 2001). The human skin contains two types of sweat glands: eccrine and apocrine. Eccrine sweat glands, found all over the body, are responsive to body temperature and release a clear liquid used to cool the body. This homeostatic mechanism occurs especially during physical activity. Apocrine sweat glands are associated with hair follicles and consequently are most commonly found in areas such as the underarms and groin. Not responsive to changes in body temperature, apocrine sweat glands are often associated with emotional/hormonal causes. Although the initial secretion of the apocrine sweat glands is odorless, it contains organic compounds in which hydrolysis by bacteria on the skin lead to body malodor.

Deodorants and antiperspirants, although commonly thought of as the same toiletry commodity, differ in their mechanism of action to help with underarm personal care management. For example, while deodorants and antiperspirants each address the effects of the apocrine and eccrine sweat glands, they are not interchangeable in their function. Antiperspirants, considered OTC (Over-the-Counter) drugs by the FDA and regulated as such, contain inorganic polymers, commonly aluminum compounds, which function by diffusing into the sweat gland and therefore blocking sweat from reaching the skin surface. Antiperspirants are not designed to control body odor, but the wetness associated with sweating. Deodorants on the other hand, are much more focused on the negative effects of the apocrine sweat glands and work to mask and potentially reduce body malodor. For example, often deodorants use fragrances to mask the body malodor. However, as body malodor is a reaction of the microflora of the skin and the sweat, deodorants can also apply antimicrobial actives into the formulation to help to reduce body odor. Examples include the use of alcohol to kill the bacteria and inhibition of hydrolysis with ingredients such as triethyl citrate.

As body odor and sweating are often considered undesirable by consumers, deodorants and antiperspirants are crucial personal care products where innovation is necessary for more efficacious and naturally-based products.

### SUMMARY

Provided herein are dermatologically acceptable compositions comprising: A) one or more of: a yeast component; a Bacillaceae extract; and 13-methyltetradecanoic acid; B) one or more of: a phytochemical; an organic acid; and an antimicrobial peptide; and C) a dermatologically acceptable carrier.

Also provided herein are dermatologically acceptable compositions comprising: A) a yeast component, a Bacillaceae extract, or a combination thereof; B) one or more of: a phytochemical; an organic acid; and an antimicrobial peptide; and C) a dermatologically acceptable carrier.

Also provided herein are dermatologically acceptable compositions comprising: A) a yeast component, 13-methyltetradecanoic acid, or a combination thereof; B) one or more of: a phytochemical; an organic acid; and an antimicrobial peptide; and C) a dermatologically acceptable carrier.

Also provided herein are dermatologically acceptable compositions comprising: A) a Bacillaceae extract, 13-methyltetradecanoic acid, or a combination thereof; B) one or more of: a phytochemical; an organic acid; and an antimicrobial peptide; and C) a dermatologically acceptable carrier.

Also provided herein are dermatologically acceptable compositions comprising: a yeast component; a phytochemical; an organic acid; and a dermatologically acceptable carrier.

Also provided herein are dermatologically acceptable compositions comprising: a yeast component; a phytochemical; and a dermatologically acceptable carrier.

Also provided herein are dermatologically acceptable compositions comprising: a Bacillaceae extract, 13-methyltetradecanoic acid, or a combination thereof; a phytochemical; and a dermatologically acceptable carrier.

Provided herein are dermatologically acceptable compositions comprising: A) two or more of: a yeast extract, yeast cell wall, or combination thereof; a phytochemical; an organic acid; and an antimicrobial peptide; and B) a dermatologically acceptable carrier. Also provided herein are dermatologically acceptable compositions comprising: a yeast extract, yeast cell wall, or combination thereof; a phytochemical; an organic acid; and a dermatologically acceptable carrier. Also provided herein are dermatologically acceptable compositions comprising: a yeast extract, yeast cell wall, or combination thereof; a phytochemical; and a dermatologically acceptable carrier.

In some embodiments, the yeast extract, yeast cell wall, or combination thereof is present in an amount of at least 0.01% w/w of the composition. In some embodiments, the yeast extract, yeast cell wall, or combination thereof is present in an amount of about 0.5% to about 20% w/w of the composition. In some embodiments, the yeast extract, yeast cell wall, or combination thereof is present in an amount of at least 1% w/w of the composition. In some embodiments, the yeast extract, yeast cell wall, or combination thereof is present in an amount of about 1% to about 5% w/w of the composition.

In some embodiments, the yeast extract comprises: a Saccharomyces extract, a Candida extract, a Debaryomyces extract, a Kloeckera extract, a Kluyveromyces extract, a Geotrichum extract, a Pichia extract, a Wickerhamomyces extract, or a combination thereof. In some embodiments, the yeast extract comprises: Saccharomyces boulardii extract, Saccharomyces cerevisiae extract, Saccharomyces pastorianus extract, Candida bombicola extract, Debaryomyces hansenii extract, Kloeckera apiculate extract, Kluyveromyces thermotolerans extract, Geotrichum candidum extract, Candida intermedia extract, Kluyveromyces marxianus extract, Pichia norvegensis extract, Pichia fermentans extract, Candida tropicalis extract, Candida apicola extract, Wickerhamiella domercqiae extract, Wickerhamomyces anomalus extract, and any combinations thereof. In some embodiments, the yeast extract is Saccharomyces pastorianus extract. In some embodiments, the yeast extract is Saccharomyces boulardii extract. In some embodiments, the yeast extract is Saccharomyces cerevisiae extract. In some embodiments, the yeast extract is a Saccharomyces boulardii and Saccharomyces cerevisiae extract.

In some embodiments, the yeast cell wall comprises: a Saccharomyces cell wall, a Candida cell wall, a Debaryomyces cell wall, a Kloeckera cell wall, a Kluyveromyces cell wall, a Geotrichum cell wall, a Pichia cell wall, a Wickerhamomyces cell wall, or a combination thereof. In some embodiments, the cell wall comprises: Saccharomyces boulardii cell wall, Saccharomyces cerevisiae cell wall, Saccharomyces pastorianus cell wall, Candida bombicola cell wall, Debaryomyces hansenii cell wall, Kloeckera apiculate cell wall, Kluyveromyces thermotolerans cell wall, Geotrichum candidum cell wall, Candida intermedia cell wall, Kluyveromyces marxianus cell wall, Pichia norvegensis cell wall, Pichia fermentans cell wall, Candida tropicalis cell wall, Candida apicola cell wall, Wickerhamiella domercqiae cell wall, Wickerhamomyces anomalus cell wall, and any combinations thereof. In some embodiments, the cell wall is Saccharomyces pastorianus cell wall. In some embodiments, the cell wall is Saccharomyces boulardii cell wall. In some embodiments, the cell wall is Saccharomyces cerevisiae cell wall. In some embodiments, the cell wall is a Saccharomyces boulardii and Saccharomyces cerevisiae cell wall.

In some embodiments, the Bacillaceae extract is present in an amount of at least 0.01% w/w of the composition. In some embodiments, the Bacillaceae extract is present in an amount of about 0.5% to about 20% w/w of the composition. In some embodiments, the Bacillaceae extract is present in an amount of at least 1% w/w of the composition. In some embodiments, the Bacillaceae extract is present in an amount of about 1% to about 5% w/w of the composition.

In some embodiments, the 13-methyltetradecanoic acid is present in an amount of at least 0.01% w/w of the composition. In some embodiments, the 13-methyltetradecanoic acid is present in an amount of about 0.5% to about 20% w/w of the composition. In some embodiments, the 13-methyltetradecanoic acid is present in an amount of at least 1% w/w of the composition. In some embodiments, the 13-methyltetradecanoic acid is present in an amount of about 1% to about 5% w/w of the composition.

In some embodiments, the phytochemical is present in an amount of about 0.01% to about 10% w/w of the composition. In some embodiments, the phytochemical is present in an amount of about 0.5% to about 6% w/w of the composition. In some embodiments, the phytochemical is present in an amount of about 0.5% to about 4% w/w of the composition.

In some embodiments, the phytochemical is selected from the group consisting of: farnesol, caffeine, caffeic acid, chlorogenic acid, a derivative of chlorogenic acid, tannic acid, trigonolline, protocatechuic acid, and a combination thereof.

In some embodiments, the phytochemical is a compound extracted from Camellia sinensis and/or a plant species from the genus Coffea. In some embodiments, the phytochemical comprises caffeine. In some embodiments, caffeine is present in an amount of about 0.1% to about 2% w/w of the composition. In some embodiments, caffeine is present in an amount of about 0.25% to about 1% w/w of the composition.

In some embodiments, the phytochemical comprises farnesol. In some embodiments, the farnesol is present in an amount of about 0.00001% to about 5% w/w of the composition. In some embodiments, the farnesol is present in an amount of about 0.00001% to about 2% w/w of the composition.

In some embodiments, the farnesol is present in the composition as a component of an extract or oil from a plant tissue from a plant genus selected from the group consisting of: Cymbopogon, Prunus, Rosa, and a combination thereof. In some embodiments, the farnesol is present in the composition as a component of an extract or oil from a plant selected from the group consisting of from a plant selected from the group consisting of: Vachellia farnesiana, Matricaria chamomilla; Abelmoschus moschatus; Myrocarpus fastigiatus; Oxystigma buccholtzii Harms; Cananga odorata; Acacia farnesiana; Myroxylon balsamum var. pereirae; Polianthes tuberosa; Pimpinella anisum; Aremisia campestris, Cyclamen Persicum, Myroxylon balsamum, Cymbopogon nardus, Cymbopogon winterianus, Cymbopogon martini, Cymbopogon schoenanthus, Cymbopogon Flexuosus Oi, Cymbopogon citratus, Prunus armeniaca, Rosa damascene, Rosa damascene, and a combination thereof. In some embodiments, the farnesol is present in the composition as a component of a lemon grass extract.

In some embodiments, the composition further comprises an organic acid. In some embodiments, the organic acid is present in an amount of about 0.01% to about 5% w/w of the composition. In some embodiments, the organic acid is present in an amount of about 0.1% to about 3% w/w of the composition.

In some embodiments, the organic acid is selected from the group consisting of: citric acid, malic acid, oxalic acid, manolic acid, malic acid, acetic acid, pyruvic acid, oxalic acid, glutaric acid, fumaric acid, formic acid, lactic acid, succinic acid, α-ketoglutaric acid, and a combination thereof. In some embodiments, the organic acid is present in the composition as a component of an extract from a citrus fruit. In some embodiments, the organic acid is present in the composition as a component of an extract from a citrus fruit selected from the group consisting of: amanatsu (Citrus natsudaidai), balady citron (Citrus medica), bergamot orange (Citrus bergamia), bitter orange (Citrus x aurantium), blood orange (Citrus x sinensis), Buddha's hand (Citrus medica var. sarcodactylis), calamondin (Citrus mitis), Cam sành (Citrus reticulata x maxima), citron (Citrus medica), clementine (Citrus reticulate), Corsican citron (Citrus medica), desert lime (Citrus glauca), etrog (Citrus medica), finger lime (Citrus australasica), Florentine citron (Citrus x limonimedica), grapefruit (Citrus x paradise), greek citron (Citrus medica), hyaganatsu (Citrus tamurana), Anadomikan (Citrus x iyo), kabosu (Citrus sphaerocarpa), kaffir lime (Citrus hystrix), key lime (Citrus aurantiifolia), kinnow (Citrus nobilis x Citrus deliciosa), kiyomi (Citrus unshiu x Citrus sinensis), kumquat (Citrus japonica), lemon (Citrus limon), sweet lime (Citrus limetta), mandarin orange (Citrus reticulata), mangshanyegan (Citrus mangshanensis), meyer lemon (Citrus x meyeri), Moroccan citron (Citrus medica), chinotto (Citrus myrtifolia), orange (Citrus x sinensis), oroblanco (Citrus grandis x C. Paradisi/Citrus maxima/Citrus grandis), a papeda, Persian lime (Citrus x latifolia), pomelo (Citrus maxima or Citrus grandis), ponderosa lemon (Citrus maxima x medica), Rangpur (Citrus x limonia), round lime (Citrus australis), satsuma (Citrus unshiu), shangjuan (Citrus ichangensis x C. maxima), shonan gold (Citrus flaviculpus hort. ex Tanaka (Ōgonkan) x Citrus unshiu), sudachi (Citrus sudachi), Taiwan tangerine (Citrus x depressa), tangelo (C. reticulata x C. maxima or x C. paradise), tangerine (Citrus tangerine), tangor (C. reticulata x C. sinensis), ugli fruit (Citrus reticulata x Citrus paradise), yuzu (Citrus ichangensis x C. reticulate), and a combination thereof. In some embodiments, the organic acid is citrus limon fruit extract.

In some embodiments, the composition further comprises an antimicrobial peptide. In some embodiments, the antimicrobial peptide is selected from the group consisting of: lugdunin, human β-defensin-1 (hBD1), hBD2, hBD3, CAP18, a hepcidin, cathelicidin peptide LL-37, dermcidin (DCD-1), adrenomedullin, elafin (SKALP), and a combination thereof. In some embodiments, the antimicrobial peptide is lugdunin. In some embodiments, the lugdunin is present in an amount of about 1.5 to about 20.5 pg/mL of the composition.

Also provided herein are dermatologically acceptable compositions comprising: a yeast extract, yeast cell wall, or combination thereof; caffeine; farnesol; and one or more organic acids. Also provided herein are dermatologically acceptable compositions comprising: a yeast extract, yeast cell wall, or combination thereof; caffeine; farnesol; one or more organic acids; and lugdunin. In some embodiments, the composition further comprises a dermatologically acceptable carrier.

In some of any of the above embodiments, the dermatologically acceptable carrier is selected from the group consisting of: Maranta Arundinacea (arrowroot) root powder, a wax (e.g., ozokerite (earth wax), carnauba wax, candelilla wax, beeswax), tapioca starch, cornstarch, propanediol, water, an alcohol (e.g., ethanol), glycerin, sodium stearate, diatomaceous earth, polyglyceryl-6 caprylate, polyglyceryl-4 caprate, ethylhexylglycerin, triethyl citrate, Aloe barbadensis leaf juice, leuconostoc/radish root ferment filtrate, jojoba esters, stearyl alcohol, cellulose, silica, hydrated silica, titanium dioxide, and a combination thereof.

In some of any of the above embodiments, the dermatologically acceptable carrier comprises one or more of: a humectant, an emollient, a solubilizer and/or emulsifier, a preservative, and a matrix agent.

In some embodiments, the humectant comprises one or more of: erythritol, pentylene glycol, propanediol, sodium pyrrolidone carboxylic acid (PCA), sodium hyaluronate, betaine, glycerin, propylene glycol, a polyethylene glycol, a sugar, hexylene glycol, butylene glycol, aloe vera gel, an alpha hydroxy acid such as lactic acid, glyceryl triacetate, lithium chloride, sorbitol, xylitol, maltitol, hyaluronic acid, allantoin, urea, tremella extract, dicyanamide, sodium lactate, and sodium L-pyroglutamate urea, and pyrrolidone carboxylic acid.

In some embodiments, the emollient comprises one or more of: elastin, cetyl alcohol, a silicone, a coconut alkane, coco-caprylate/caprate, diheptyl succinate, capryloyl glycerin/sebacic acid copolymer, triethyl citrate, caprylic/capric triglyceride, a butter (e.g., Butyrospermum parkii butter (shea butter) or Theobroma cacao (cocoa) seed butter), collagen, colloidal oatmeal, elastin, glyceryl stearate, isopropyl palmitate, shea butter, coconut oil (Cocos nucifera oil), and stearic acid.

In some embodiments, the solubilizer and/or emulsifier comprises one or more of: polyglyceryl-6 caprylate and polyglyceryl-4 caprate.

In some embodiments, the matrix agent comprises one or more of: corn starch, sodium bicarbonate (baking soda), magnesium hydroxide, sodium hydroxide, Maranta Arundinacea (arrowroot) root powder, a wax (e.g., ozokerite (earth wax), carnauba wax, candelilla (Euphorbia cerifera) wax, beeswax, microcrystalline wax, and Oryza sativa (rice) bran wax), tapioca starch, cornstarch, propanediol, leuconostoc/radish root ferment, water, sodium stearate, diatomaceous earth, cellulose, silica, hydrated silica, and titanium dioxide.

In some embodiments, the preservative is selected from the group consisting of: leuconostoc/radish root ferment filtrate, a benzoate, a benzoic acid, a propionate, propionic acid, a sorbate, sorbic acid, a salicylic acid, a salicylate, hexa-2,4-dienoic acid, a hexa-2,4-dienoate, formic acid, 3-acetyl-6-methylpyran-2,4-(3H)-dione, 3,3′-dibromo-4,4′-hexamethylenedioxydibenzamidine and its salts, thiomersal, phenylmercuric salts, undec-10-enoic acid and its salts, 1,3-bis (2-ethylhexyl) hexahydro-5-methyl-5-pyrimidine, 5-bromo-5-nitro-1,3-dioxane, bronopol, 2,4-dichlorobenzyl alcohol, 1-(4-chlorophenyl)-3-(3,4-dichlorophenyl) urea, chlorocresol, chloroxylenol, 5-chloro-2-(2,4-dichlorophenoxy) phenol, N,N″-methylenebis[N′-[3-(hydroxymethyl)-2,5-dioxoimidazolidin-4-yl]urea], polyaminopropyl biguanide, methenamine, quaternium-15, climbazole, DMDM hydantoin, benzyl alcohol, 1-Hydroxy-4-methyl-6-(2,4,4-trimethylpentyl)-2-pyridon, piroctone olamine, bromochlorophene, o-cymen-5-ol, chlorophene, chloroacetaminde, methylchloroisothiazolinone, methylisothiazolinone, phenoxyisopropanol, chlorhexidine, chlorhexidine diacetate, chlorhexidine digluconate, chlorhexidine dihydrochloride, dimethyl oxazolidine, behentrimonium chloride, cetrimonium bromide, cetrimonium chloride, laurtrimonium bromide, laurtrimonium chloride, steartrimonium bromide, steartrimonium chloride, diazolidinyl urea, hexamidine, hexamidine diisethionate, hexamidine paraben, glutaral, 7-ethylbicyclooxazolidine, chlorphenesin, sodium hydroxymethylglycinate, silver chloride, benzethonium chloride, benzalkonium chloride, benza-lkonium bromide, benzalkonium saccharinate, benzylhemiformal, iodopropynyl butylcarbamate, biphenyl-2-ol and its salts, pyrithionine zinc, an erythorbate, a nitrite, ethylenediaminetetraacetic acid (EDTA), butylated hydroxyanisole (BHA), butylated hydroxytoluene (BHT), capryllic acid, dilauryl thiodipropionate, erythorbic acid, gum guaiac, methylparaben, a sulfite, a bisulfite, a metabisulfite, propyl gallatepy, propylparaben, stannous chloride, sulfur dioxide, thiodipropionic acid, an isothiazoline, a paraben, phenoxyethanol, ethylhexylglycerin, a glycol, caprylhydroxamic acid, caprylyl glycol, glyceryl capylate, sodium benzoate, potassium sorbate, 1,2-hexanediol, propanediol, leuconostoc/radish root ferment, and a tocopherol.

In some embodiments, the dermatologically acceptable carrier comprises propanediol, water, glycerin, stearic acid, and sodium hydroxide. In some embodiments, the dermatologically acceptable carrier comprises propanediol, water, glycerin, stearic acid, sodium hydroxide, silica, and hydrated silica. In some embodiments, the dermatologically acceptable carrier further comprises one or more of: diatomaceous earth, Maranta arundinacea (arrowroot) root powder, polyglyceryl-6 caprylate, polyglyceryl-4 caprate, leuconostoc/radish root ferment filtrate, Aloe barbadensis leaf juice, jojoba esters, cellulose, ethylhexylglycerin, triethyl citrate, stearyl alcohol, and titanium dioxide.

In some embodiments, the composition further comprises an antiperspirant. In some embodiments, the antiperspirant comprises one or more of: aluminum chloride, aluminum chlorohydrate, aluminum chlorohydrex polyethylene glycol complex, aluminum chlorohydrex propylene glycol complex, aluminum dichlorohydrate, aluminum dichlorohydrex polyethylene glycol complex, aluminum dichlorohydrex propylene glycol complex, aluminum sesquichlorohydrate, aluminum sesquichlorohydrex polyethylene glycol complex, aluminum sesquichlorohydrex propylene glycol complex, aluminum sulfate buffered with sodium aluminum lactate, aluminum zirconium octachlorohydrate, aluminum zirconium octachlorohydrex glycine complex, aluminum zirconium pentachlorohydrate, aluminum zirconium pentachlorohydrex glycine complex, aluminum zirconium tetrachlorohydrate, aluminum zirconium tetrachlorohydrex glycine complex, aluminum zirconium trichlorohydrate, and aluminum zirconium trichlorohydrex glycine complex. In some embodiments, the composition is configured for topical application. In some embodiments, the composition is in the form of a solid, a semi-solid, a lotion, an emulsion, or a liquid (e.g., a spray or roll-on).

Also provided herein are methods for reducing body odor comprising applying a composition of as described herein. Also provided herein are methods for reducing the production of one or more malodorous compounds by bacteria in sweat comprising applying a composition as described herein.

In some embodiments, the one or more malodorous compounds are selected from the group consisting of: a thioalcohol, acetic acid, isovaleric acid, 3-methyl-2-hexenoic acid, propionic acid, and 3-hydroxy-3-methylhexanoic acid.

In some embodiments, the bacteria are from a genus selected from the group consisting of: Corynebacterium, Micrococcus, Staphylococcus, Propionibacterium, Brevibacterium, Cutibacterium, and a combination thereof. In some embodiments, the bacteria are from a species selected from the group consisting of: S. hominis, S. aureus, S. epidermidis, Cutibacterium avidum, and a combination thereof.

In some embodiments, the composition is applied to the subject on an area selected from the group consisting of: an underarm, the face, a heel of a foot, a hairline, an inner thigh, an area behind a knee, a sole of a foot, a hand, the groin, genitalia, perineum and a combination thereof.

In some embodiments, the composition is applied daily. In some embodiments, the composition is applied once every two days or once every three days.

### DETAILED DESCRIPTION

Skin, as the largest organ of the human body, is host to an abundance of bacteria, viruses, and fungi that comprise the skin microbiome. The skin microflora can function symbiotically with the skin and can assist with vital functions, such as immune response against pathogens (see, e.g., Byrd et al. Nat. Rev. Microbiol. 2018; 16(3):143-155). The skin microbiome of the human axilla is not only abundant, but also diverse, and the connection between body malodor and the skin microflora has been linked for over 50 years (see Minhas et al. Elife. 2018;7. pii: e34995). Corynebacterium, Micrococcus, Staphylococcus, Propionibacterium, and Brevibacterium have all been found to flourish in the human axillary region, with Corynebacterium ssp. and Staphylococcus ssp. being the most abundant (see Callewaert et al. PLoS One. 2013; 8(8):e70538). S. hominis can convert the precursor compounds in sweat into thioalcohols, the chemicals most noted for their strong malodor (see Minhas et al. Elife. 2018;7. pii: e34995). Additional bacteria that can result in body malodor include Staphylococcus epidermidis, which can lead to the production of sour odor-associated chemicals, such as acetic acid and isovaleric acid (Lam et al. Microbiome. 2018; 6(1):213). Corynebacterium ssp. can also produce strong body malodor and can produce a known foul-odor chemical called 3-methyl-2-hexenoic acid (see Natsch et al. J. Biol. Chem. 2003; 278(8):5718-27). As the microflora can be essential in the production of the smelly chemical compounds in the axillary region, helping to prevent the production of these compounds by antimicrobial methods is an effective method to help reduce body odor.

Accordingly, the present application provides topical compositions comprising A) one or more of: a yeast component (e.g., a yeast extract, yeast cell wall, or combination thereof); a Bacillaceae extract; and 13-methyltetradecanoate; and B) one or more of: a phytochemical; an organic acid; and an antimicrobial peptide, Such topical compositions are useful in the reduction of body odors.

### Definitions

As used herein, the phrases an “effective amount” or a “dermatologically effective amount” of an active agent or ingredient, refer to an amount of the active agent sufficient to reduce or eliminate one or more symptoms of a condition. Effective amounts of the dermatologically active agent will vary with the kind of dermatologically active agent chosen, the severity of the condition, the specific components of the composition being used, and like factors. For example, the presently described compositions can be topically applied in an amount sufficient to cover an area of interest, e.g., a sweaty area or an area that can become sweaty, plus a margin of skin or tissue surrounding the area of interest, for example, a margin of about 0.5 inches, at a frequency, for example, of once a day.

The term “yeast” or “yeast cell” refers to a phylogenetically diverse group of single-celled fungi, most of which are in the division of Ascomycota and Basidiomycota.

As used herein, “subject” refers to any subject, particularly a mammalian subject for example, a human. For example, a subject can refer to a mammalian subject, e.g., a human, for whom a reduction in body odor is desired.

Reference to the term “about” has its usual meaning in the context of compositions to allow for reasonable variations in amounts that can achieve the same effect and also refers herein to a value of plus or minus 10% of the provided value. For example, “about 20” means or includes amounts from 18 to and including 22.

Unless otherwise defined, all technical and scientific terms used herein have the same meaning as commonly understood by one of ordinary skill in the art to which this invention belongs. In case of conflict, the present specification, including definitions, will control. Throughout this specification and claims, the word “comprise,” or variations such as “comprises” or “comprising” will be understood to imply the inclusion of a stated integer or group of integers but not the exclusion of any other integer or group of integers. Unless otherwise required by context, singular terms shall include pluralities and plural terms shall include the singular. As used herein, the singular form “a”, “an”, and “the” include plural references unless indicated otherwise. For example, “an” excipient includes one or more excipients. It is understood that aspects and variations of the invention described herein include “consisting of” and/or “consisting essentially of” aspects and variations. Methods and materials are described herein for use in the present invention; other, suitable methods and materials known in the art can also be used. The materials, methods, and examples are illustrative only and not intended to be limiting. All publications, patent applications, patents, sequences, databases entries, and other references mentioned herein are incorporated by reference in their entirety.

### Compositions

Provided herein are dermatologically acceptable compositions comprising: A) one or more of: a yeast component (e.g., a yeast extract, yeast cell wall, or combination thereof); a Bacillaceae extract; and 13-methyltetradecanoic acid; B) one or more of: a phytochemical; an organic acid; and an antimicrobial peptide; and C) a dermatologically acceptable carrier. In some embodiments, a composition as described herein comprises: A) a yeast component (e.g., a yeast extract, yeast cell wall, or combination thereof); B) one or more of: a phytochemical; an organic acid; and an antimicrobial peptide; and C) a dermatologically acceptable carrier. In some embodiments, a composition as described herein comprises: A) a Bacillaceae extract; B) one or more of: a phytochemical; an organic acid; and an antimicrobial peptide; and C) a dermatologically acceptable carrier. In some embodiments, a composition as described herein comprises: A) 13-methyltetradecanoic acid; B) one or more of: a phytochemical; an organic acid; and an antimicrobial peptide; and C) a dermatologically acceptable carrier. In some embodiments, a composition as described herein comprises: A) a yeast component (e.g., a yeast extract, yeast cell wall, or combination thereof) and a Bacillaceae extract; B) one or more of: a phytochemical; an organic acid; and an antimicrobial peptide; and C) a dermatologically acceptable carrier. In some embodiments, a composition as described herein comprises: A) a yeast component (e.g., a yeast extract, yeast cell wall, or combination thereof) and 13-methyltetradecanoic acid; B) one or more of: a phytochemical; an organic acid; and an antimicrobial peptide; and C) a dermatologically acceptable carrier. In some embodiments, a composition as described herein comprises: A) a Bacillaceae extract and 13-methyltetradecanoic acid; B) one or more of: a phytochemical; an organic acid; and an antimicrobial peptide; and C) a dermatologically acceptable carrier.

In some embodiments, a composition as described herein comprises: A) a yeast component (e.g., a yeast extract, yeast cell wall, or combination thereof); B) a phytochemical; and C) a dermatologically acceptable carrier. In some embodiments, a composition as described herein comprises: A) a yeast component (e.g., a yeast extract, yeast cell wall, or combination thereof); B) a phytochemical; and an organic acid; and C) a dermatologically acceptable carrier. In some embodiments, a composition as described herein comprises: A) a yeast component (e.g., a yeast extract, yeast cell wall, or combination thereof); B) a phytochemical; and an antimicrobial peptide; and C) a dermatologically acceptable carrier. In some embodiments, a composition as described herein comprises A) a yeast component (e.g., a yeast extract, yeast cell wall, or combination thereof); B) an organic acid; and an antimicrobial peptide; and C) a dermatologically acceptable carrier. In some embodiments, a composition as described herein comprises A) a yeast component (e.g., a yeast extract, yeast cell wall, or combination thereof); B) a phytochemical; an organic acid; and an antimicrobial peptide; and C) a dermatologically acceptable carrier.

Also provided herein are dermatologically acceptable compositions comprising: a yeast component (e.g., a yeast extract, yeast cell wall, or a combination thereof) and a phytochemical. In some embodiments the dermatologically acceptable composition comprises: a yeast component (e.g., a yeast extract, yeast cell wall, or a combination thereof); a phytochemical; and an organic acid.

Also provided herein are dermatologically acceptable compositions comprising: A) one or more of: a yeast component (e.g., a yeast extract, yeast cell wall, or combination thereof); a phytochemical; an organic acid; and an antimicrobial peptide; and B) a dermatologically acceptable carrier. In some embodiments, a dermatologically acceptable composition as described herein comprises: A) two or more of: a yeast component (e.g., a yeast extract, yeast cell wall, or combination thereof); a phytochemical; an organic acid; and an antimicrobial peptide; and B) a dermatologically acceptable carrier. In some embodiments, a dermatologically acceptable composition as described herein comprises: A) three or more of: a yeast component (e.g., a yeast extract, yeast cell wall, or combination thereof); a phytochemical; an organic acid; and an antimicrobial peptide; and B) a dermatologically acceptable carrier. In some embodiments, a dermatologically acceptable composition as described herein comprises: A) four or more of: a yeast component (e.g., a yeast extract, yeast cell wall, or combination thereof); a phytochemical; an organic acid; and an antimicrobial peptide; and B) a dermatologically acceptable carrier.

In some embodiments, a composition as described herein comprises A) a phytochemical; an organic acid; and an antimicrobial peptide; and B) a dermatologically acceptable carrier.

A “yeast component” as used herein refers to one or more components of a ruptured yeast cell. For example, a yeast component can include all or portions of a yeast cell wall, periplasm, cell membrane, cytoplasm, nucleus, or a combination thereof. In some embodiments, a yeast component comprises a yeast extract, a yeast cell wall, or a combination thereof.

A “yeast extract” as used herein refers to one or more cytoplasmic or nuclear components of a ruptured yeast cell. For example, a yeast extract can include all or portions of the cytoplasm and/or nucleus from a ruptured yeast cell. In some embodiments, a yeast extract includes cytosol and one or more organelles. In some embodiments, a yeast extract includes one or more amino acids, proteins, peptides, nucleic acids, minerals, flavonoids, sugars, lipids, and vitamins from the yeast cell (e.g., from the yeast cytoplasm, the yeast nucleus, or both). In some embodiments, a yeast extract includes cytosol, any organelle, enzymes, metalloproteases, cysteine proteases, aspartic proteases, serine proteases, proteins, carbohydrates, lipids, chitins, proteases, or a combination thereof. In some embodiments, a yeast extract is a yeast supernatant, yeast filtrate, yeast cell product, yeast cytoplasmic product, or a combination thereof. A yeast extract can be prepared by any of the methods known to one of ordinary skill in the art. For example, a yeast extract can prepared by autolysing yeast and separating the cell wall from the soluble portion, e.g., the yeast extract. A yeast extract can be extracted from, isolated from, and/or prepared from one or more species of yeast. Non-limiting examples of yeast genuses from which a yeast species can be used to produce a yeast extract include: Saccharomyces, Candida, Debaryomyces, Kloeckera, Kluyveromyces, Geotrichum, Pichia, and Wickerhamomyces. Non-limiting examples of yeast species that can be used to produce a yeast extract include: Saccharomyces boulardii, Saccharomyces cerevisiae, Saccharomyces pastorianus, Candida bombicola, Debaryomyces hansenii, Kloeckera apiculate, Kluyveromyces thermotolerans, Geotrichum candidum, Candida intermedia, Kluyveromyces marxianus, Pichia norvegensis, Pichia fermentans, Candida tropicalis, Candida apicola, Wickerhamiella domercqiae, and Wickerhamomyces anomalus. As used herein, “a yeast extract” can also be referred to as a “yeast essence” and “yeast ferment filtrate.” For example, “Saccharomyces extract” can also be referred to as “Saccharomyces ferment filtrate.”

In some embodiments, a yeast extract can decrease or inhibit one or more of circulating bacterial numbers, bacterial function, bacterial growth, bacterial toxin elaboration, bacterial recruitment, bacterial reproduction, bacterial viability, and bacterial migration. In some embodiments, a yeast extract can inhibit bacterial pathogenic response.

“Yeast cell wall” as used herein refers to yeast cell wall components separated from the yeast cytoplasm and nucleus. Yeast cell wall can include, for example, fragments of the cell wall and/or any protein, carbohydrate, or other derivative of the cell wall. In some embodiments, yeast cell wall includes mannan and/or a beta-glucan from the cell wall of the yeast. Yeast cell wall can be prepared by any of the methods known to one of ordinary skill in the art. For example, insoluble yeast cell wall components can be separated (e.g., by centrifugation) from soluble yeast extract. As another example, yeast cell wall can be obtained from hydrolysis of yeast cell walls under controlled conditions, and the hydrolyzed cell walls can be purified from other yeast cell components by centrifugation. Yeast cell walls can also be spray-dried after centrifugation. Yeast cell wall can be prepared from one or more species of yeast. Non-limiting examples of yeast genuses from which a yeast species can be used to produce yeast cell wall include: Saccharomyces, Candida, Debaryomyces, Kloeckera, Kluyveromyces, Geotrichum, Pichia, and Wickerhamomyces. Non-limiting examples of yeast species that can be used to produce yeast cell wall include: Saccharomyces boulardii, Saccharomyces cerevisiae, Saccharomyces pastorianus, Candida bombicola, Debaryomyces hansenii, Kloeckera apiculate, Kluyveromyces thermotolerans, Geotrichum candidum, Candida intermedia, Kluyveromyces marxianus, Pichia norvegensis, Pichia fermentans, Candida tropicalis, Candida apicola, Wickerhamiella domercqiae, and Wickerhamomyces anomalus.

In some embodiments, the yeast component (e.g., yeast extract, yeast cell wall, or combination thereof) is present in an amount of at least about 0.01% w/w of the composition. For example, at least about 0.1%, about 0.5%, about 1%, about 2%, about 3%, about 5%, about 10%, or about 20% w/w of the composition. In some embodiments, the yeast component is present in an amount of about 0.01% to about 25% w/w of the composition. For example, about 0.01% to about 0.1%, about 0.01% to about 0.25%, about 0.01% to about 0.5%, about 0.01% to about 1%, about 0.01% to about 2%, about 0.01% to about 3%, about 0.01% to about 4%, about 0.01% to about 5%, about 0.01% to about 6%, about 0.01% to about 7%, about 0.01% to about 8%, about 0.01% to about 9%, about 0.01% to about 10%, about 0.01% to about 15%, about 0.01% to about 20%, about 20% to about 25%, about 15% to about 10%, about 9% to about 25%, about 8% to about 25%, about 7% to about 25%, about 6% to about 25%, about 5% to about 25%, about 4% to about 25%, about 3% to about 25%, about 2% to about 25%, or about 1% to about 25% w/w of the composition. In some embodiments, the yeast component is present in an amount of about 1% to about 5%, about 2% to about 7%, about 5% to about 10%, about 10% to about 15%, about 1% to about 10%, or about 5% to about 15% w/w of the composition. In some embodiments, the yeast component is present in an amount of about 0.01% to about 0.3%, about 0.1% to about 0.3%, about 0.1% to about 0.3%, about 0.2% to about 0.5%, about 0.3% to about 0.5%, about 0.4% to about 0.6%, about 0.5% to about 0.7%, about 0.6% to about 0.8%, about 0.7% to about 0.9%, about 0.8% to about 1%, about 0.9% to about 1.1%, about 1% to about 1.2%, about 1.1% to about 1.3%, about 1.2% to about 1.4%, about 1.3% to about 1.5%, about 1.4% to about 1.6%, about 1.5% to about 1.7%, about 1.6% to about 1.8%, about 1.7% to about 1.9%, about 1.8% to about 2%, about 2% to about 2.5%, about 2.5% to about 3%, about 3% to about 3.5%, or about 3.5% to about 4% w/w of the composition. In some embodiments, the yeast component is present in an amount of about 0.01%, about 0.1%, about 0.2%, about 0.25%, about 0.3%, about 0.35%, about 0.4%, about 0.45%, about 0.5%, about 0.6%, about 0.7%, about 0.8%, about 0.9%, about 1%, about 1.2%, about 1.4%, about 1.6%, about 1.8%, about 2%, about 2.2%, about 2.4%, about 2.6%, about 2.8%, about 3%, about 3.5%, about 4%, about 4.5%, about 5%, about 5.5%, about 6%, about 6.5%, about 7%, about 7.5%, about 8%, about 9%, about 10%, about 15%, or about 20% w/w of the composition.

In some embodiments, the yeast component is selected from the group consisting of: a Saccharomyces component, a Candida component, a Debaryomyces component, a Kloeckera component, a Kluyveromyces component, a Geotrichum component, a Pichia component, a Wickerhamomyces component, and a combination thereof. In some embodiments, the Saccharomyces component, the Candida component, the Debaryomyces component, the Kloeckera component, the Kluyveromyces component, the Geotrichum component, the Pichia component, and/or the Wickerhamomyces component consist of one yeast species or more than one yeast species. In some embodiments, the yeast component is selected from the group consisting of: Saccharomyces boulardii component, Saccharomyces cerevisiae component, Saccharomyces pastorianus component, Candida bombicola component, Debaryomyces hansenii component, Kloeckera apiculate component, Kluyveromyces thermotolerans component, Geotrichum candidum component, Candida intermedia component, Kluyveromyces marxianus component, Pichia norvegensis component, Pichia fermentans component, Candida tropicalis component, Candida apicola component, Wickerhamielladomercqiae component, Wickerhamomyces anomalus component, and a combination thereof. In some embodiments, the yeast component is a yeast component from a yeast species selected from the group consisting of: Saccharomyces boulardii, Saccharomyces cerevisiae, Saccharomyces pastorianus, Candida bombicola, and a combination thereof.

In some embodiments, the yeast component comprises a yeast extract. In some embodiments, the yeast extract is selected from the group consisting of: a Saccharomyces extract, a Candida extract, a Debaryomyces extract, a Kloeckera extract, a Kluyveromyces extract, a Geotrichum extract, a Pichia extract, a Wickerhamomyces extract, and a combination thereof. In some embodiments, the Saccharomyces extract, the Candida extract, the Debaryomyces extract, the Kloeckera extract, the Kluyveromyces extract, the Geotrichum extract, the Pichia extract, and/or the Wickerhamomyces extract consist of one yeast species or more than one yeast species. In some embodiments, the yeast extract is selected from the group consisting of: Saccharomyces boulardii extract, Saccharomyces cerevisiae extract, Saccharomyces pastorianus extract, Candida bombicola extract, Debaryomyces hansenii extract, Kloeckera apiculate extract, Kluyveromyces thermotolerans extract, Geotrichum candidum extract, Candida intermedia extract, Kluyveromyces marxianus extract, Pichia norvegensis extract, Pichia fermentans extract, Candida tropicalis extract, Candida apicola extract, Wickerhamielladomercqiae extract, Wickerhamomyces anomalus extract, and a combination thereof. In some embodiments, the yeast extract is an extract from a yeast species selected from the group consisting of: Saccharomyces boulardii, Saccharomyces cerevisiae, Saccharomyces pastorianus, Candida bombicola, and a combination thereof.

In some embodiments, the yeast component comprises a yeast cell wall. In some embodiments, the yeast cell wall is selected from the group consisting of: a Saccharomyces cell wall, a Candida cell wall, a Debaryomyces cell wall, a Kloeckera cell wall, a Kluyveromyces cell wall, a Geotrichum cell wall, a Pichia cell wall, a Wickerhamomyces cell wall, and a combination thereof. In some embodiments, the Saccharomyces cell wall, the Candida cell wall, the Debaryomyces cell wall, the Kloeckera cell wall, the Kluyveromyces cell wall, the Geotrichum cell wall, the Pichia cell wall, and/or the Wickerhamomyces cell wall consist of one yeast species or more than one yeast species. In some embodiments, the yeast cell wall is selected from the group consisting of: Saccharomyces boulardii cell wall, Saccharomyces cerevisiae cell wall, Saccharomyces pastorianus cell wall, Candida bombicola cell wall, Debaryomyces hansenii cell wall, Kloeckera apiculate cell wall, Kluyveromyces thermotolerans cell wall, Geotrichum candidum cell wall, Candida intermedia cell wall, Kluyveromyces marxianus cell wall, Pichia norvegensis cell wall, Pichia fermentans cell wall, Candida tropicalis cell wall, Candida apicola cell wall, Wickerhamiella domercqiae cell wall, Wickerhamomyces anomalus cell wall, and a combination thereof. In some embodiments, the yeast cell wall is Saccharomyces pastorianus cell wall. In some embodiments, the yeast cell wall is Saccharomyces boulardii cell wall. In some embodiments, the yeast cell wall is Saccharomyces cerevisiae cell wall. In some embodiments, the yeast cell wall is a Saccharomyces boulardii and Saccharomyces cerevisiae cell wall.

In some embodiments, the yeast component (e.g., yeast extract, yeast cell wall, or combination thereof) is prepared from one or more species of yeast belonging to the genus Saccharomyces.

In some embodiments, the yeast component is from one or more species of yeast belonging to the genus Saccharomyces (also referred to herein as a “Saccharomyces yeast component” or “Saccharomyces component”). In some embodiments, the yeast component is a Saccharomyces component. In some embodiments, the Saccharomyces component (e.g., Saccharomyces extract, cell wall, or combination thereof), is present in an amount of at least about 0.01% w/w of the composition. For example, at least about 0.1%, about 0.5%, about 1%, about 2%, about 3%, about 5%, about 10%, or about 20% w/w of the composition. In some embodiments, the Saccharomyces component (e.g., Saccharomyces extract, Saccharomyces cell wall, or combination thereof), is present in an amount of about 0.01% to about 25% w/w of the composition. For example, about 0.01% to about 0.5%, about 0.01% to about 1%, about 0.01% to about 2%, about 0.01% to about 3%, about 0.01% to about 4%, about 0.01% to about 5%, about 0.01% to about 6%, about 0.01% to about 7%, about 0.01% to about 8%, about 0.01% to about 9%, about 0.01% to about 10%, about 0.01% to about 15%, about 0.01% to about 20%, about 20% to about 25%, about 20% to about 25%, about 15% to about 10%, about 9% to about 25%, about 8% to about 25%, about 7% to about 25%, about 6% to about 25%, about 5% to about 25%, about 4% to about 25%, about 3% to about 25%, about 2% to about 25%, or about 1% to about 25% w/w of the composition. In some embodiments, the Saccharomyces component (e.g., Saccharomyces extract, Saccharomyces cell wall, or combination thereof), is present in an amount of about 0.01% to about 0.3%, about 0.1% to about 0.3%, about 0.2% to about 0.5%, about 0.3% to about 0.5%, about 0.4% to about 0.6%, about 0.5% to about 0.7%, about 0.6% to about 0.8%, about 0.7% to about 0.9%, about 0.8% to about 1%, about 0.9% to about 1.1%, about 1% to about 1.2%, about 1.1% to about 1.3%, about 1.2% to about 1.4%, about 1.3% to about 1.5%, about 1.4% to about 1.6%, about 1.5% to about 1.7%, about 1.6% to about 1.8%, about 1.7% to about 1.9%, about 1.8% to about 2%, about 2% to about 2.5%, about 2.5% to about 3%, about 3% to about 3.5%, or about 3.5% to about 4% w/w of the composition. For example, about 0.01%, about 0.1%, about 0.2%, about 0.3%, about 0.4%, about 0.5%, about 0.6%, about 0.7%, about 0.8%, about 0.9%, about 1%, about 1.2%, about 1.4%, about 1.6%, about 1.8%, about 2%, about 2.2%, about 2.4%, about 2.6%, about 2.8%, about 3%, about 3.5%, or about 4% w/w of the composition.

In some embodiments, the yeast component is from Saccharomyces boulardii. In some embodiments, the yeast component is from Saccharomyces cerevisiae. In some embodiments, the yeast component is from Saccharomyces pastorianus. In some embodiments, the yeast component is from Saccharomyces boulardii and Saccharomyces cerevisiae.

In some embodiments, the Saccharomyces yeast component comprises a Saccharomyces yeast extract, a Saccharomyces yeast cell wall, or a combination thereof. In some embodiments, the Saccharomyces yeast extract is an extract from one or more species of yeast belonging to the genus Saccharomyces (also referred to herein as a “Saccharomyces yeast extract” or “Saccharomyces extract”). In some embodiments, the Saccharomyces yeast cell wall is a cell wall from one or more species of yeast belonging to the genus Saccharomyces (also referred to herein as a “Saccharomyces yeast cell wall” or “Saccharomyces cell wall”).

In some embodiments, the Saccharomyces yeast extract, Saccharomyces yeast cell wall, or combination thereof is prepared from Saccharomyces boulardii. In some embodiments, the Saccharomyces yeast extract, Saccharomyces yeast cell wall, or combination thereof is prepared from Saccharomyces pastorianus. In some embodiments, the Saccharomyces yeast extract, Saccharomyces yeast cell wall, or combination thereof is prepared from Saccharomyces cerevisiae. In some embodiments, the Saccharomyces yeast extract, Saccharomyces yeast cell wall, or combination thereof is prepared from Saccharomyces boulardii and Saccharomyces cerevisiae. In some embodiments, the Saccharomyces yeast extract is an extract from Saccharomyces boulardii. In some embodiments, the Saccharomyces yeast extract is an extract from Saccharomyces cerevisiae. In some embodiments, the Saccharomyces yeast extract is an extract from Saccharomyces pastorianus. In some embodiments, the Saccharomyces yeast extract is an extract from Saccharomyces boulardii and Saccharomyces cerevisiae. In some embodiments, the Saccharomyces yeast cell wall is a cell wall from Saccharomyces boulardii. In some embodiments, the Saccharomyces yeast cell wall is a cell wall from Saccharomyces cerevisiae. In some embodiments, the Saccharomyces yeast cell wall is a cell wall from Saccharomyces pastorianus. In some embodiments, the Saccharomyces yeast cell wall is a cell wall from Saccharomyces boulardii and Saccharomyces cerevisiae.

In some embodiments, a composition described herein comprises an intact yeast cell. In some embodiments, the intact yeast cell is a live yeast cell. An intact yeast cell can be of any of the yeasts described herein.

In some embodiments, a composition described herein comprises a Bacillaceae extract. A “Bacillaceae extract” as used herein refers to one or more components of a ruptured Bacillaceae cell. For example, a Bacillaceae extract can include all or portions of cytoplasm. In some embodiments, a Bacillaceae extract can include, for example, one or more amino acids, proteins, peptides, nucleic acids, minerals, flavonoids, sugars, lipids, and vitamins from the Bacillaceae cell. In some embodiments, a Bacillaceae extract can include the cytosol, enzymes, metalloproteases, cysteine proteases, aspartic proteases, serine proteases, proteins, carbohydrates, lipids, chitins, or proteases. In some embodiments, a Bacillaceae extract can be a Bacillaceae supernatant, Bacillaceae filtrate, Bacillaceae cell product, Bacillaceae cytoplasmic product, or a combination thereof. A Bacillaceae extract can be prepared by any of the methods known to one of ordinary skill in the art. For example, a yeast extract can prepared by lysing Bacillaceae and separating all or a portion of the cell wall from the soluble portion, e.g., the Bacillaceae extract. A Bacillaceae extract can be extracted from, isolated from, and/or prepared from one or more genuses of Bacillaceae bacteria such as Bacillus and Anoxybacillus. Non-limiting examples of Bacillaceae extracts include: an Anoxybacillus kamchatkensis extract, a Bacillus subtilis extract, and a Bacillus coagulans extract. As used herein, “a Bacillaceae extract” can also be referred to as a “Bacillaceae ferment” and “Bacillaceae ferment filtrate.” For example, “Bacillus extract” can also be referred to as “Bacillus ferment filtrate” or “Bacillus ferment.” In some embodiments, the Bacillaceae extract is an Anoxybacillus kamchatkensis extract. In some embodiments, the Bacillus extract and/or Anoxybacillus kamchatkensis extract is present in the composition in as part of a combination with a vegetable oil (e.g., Simmondsia Chinensis Seed Oil (and) Avena Sativa Kernel Oil (and) Bacillus Ferment; BIOME OLEOACTIF®).

In some embodiments, the Bacillaceae extract increases RNase7 gene expression (e.g., in human skin cells contacted with Bacillaceae extract).

In some embodiments, the Bacillaceae extract is present in an amount of about 0.01% to about 20% w/w of the composition. For example, about 0.01% to about 0.5%, about 0.01% to about 1%, about 0.01% to about 2%, about 0.01% to about 3%, about 0.01% to about 4%, about 0.01% to about 5%, about 0.01% to about 6%, about 0.01% to about 7%, about 0.01% to about 8%, about 0.01% to about 9%, about 0.01% to about 10%, about 0.01% to about 15%, about 0.01% to about 18%, about 18% to about 20%, about 15% to about 20%, about 9% to about 20%, about 8% to about 20%, about 7% to about 20%, about 6% to about 20%, about 5% to about 20%, about 4% to about 20%, about 3% to about 20%, about 2% to about 20%, or about 1% to about 20% w/w of the composition. In some embodiments, the Bacillaceae extract is present in an amount of about 1% to about 10% w/w, about 5% to about 15%, about 1% to about 5%, about 5% to about 10% w/w, or about 10% to about 15% w/w of the composition. In some embodiments, the Bacillaceae extract is present in an amount of about 1% to about 5% w/w of the composition. In some embodiments, the Bacillaceae extract is present in an amount of about 0.01% to about 0.3%, about 0.1% to about 0.3%, about 0.2% to about 0.5%, about 0.3% to about 0.5%, about 0.4% to about 0.6%, about 0.5% to about 0.7%, about 0.6% to about 0.8%, about 0.7% to about 0.9%, about 0.8% to about 1%, about 0.9% to about 1.1%, about 1% to about 1.2%, about 1.1% to about 1.3%, about 1.2% to about 1.4%, about 1.3% to about 1.5%, about 1.4% to about 1.6%, about 1.5% to about 1.7%, about 1.6% to about 1.8%, about 1.7% to about 1.9%, about 1.8% to about 2%, about 2% to about 2.5%, about 2.5% to about 3%, about 3% to about 3.5%, or about 3.5% to about 4% w/w of the composition. In some embodiments, the Bacillaceae extract is present in an amount of about 0.01%, about 0.1%, about 0.2%, about 0.3%, about 0.4%, about 0.5%, about 0.6%, about 0.7%, about 0.8%, about 0.9%, about 1%, about 1.2%, about 1.4%, about 1.6%, about 1.8%, about 2%, about 2.2%, about 2.4%, about 2.6%, about 2.8%, about 3%, about 3.5%, about 4%, about 5%, about 6%, about 7%, about 8%, about 9%, about 10%, about 11%, about 12%, about 13%, about 14%, about 15%, about 16%, about 17%, about 18%, about 19%, or about 20% w/w of the composition. In some embodiments, the Bacillaceae extract is an Anoxybacillus kamchatkensis extract.

In some embodiments, a composition as described herein comprises 13-methyltetradecanoate. 13-methyltetradecanoate is also referred to as “iso C15 fatty acid.”

In some embodiments, the 13-methyltetradecanoate is present in an amount of about 0.01% to about 20% w/w of the composition. For example, about 0.01% to about 0.5%, about 0.01% to about 1%, about 0.01% to about 2%, about 0.01% to about 3%, about 0.01% to about 4%, about 0.01% to about 5%, about 0.01% to about 6%, about 0.01% to about 7%, about 0.01% to about 8%, about 0.01% to about 9%, about 0.01% to about 10%, about 0.01% to about 15%, about 0.01% to about 18%, about 18% to about 20%, about 15% to about 20%, about 9% to about 20%, about 8% to about 20%, about 7% to about 20%, about 6% to about 20%, about 5% to about 20%, about 4% to about 20%, about 3% to about 20%, about 2% to about 20%, or about 1% to about 20% w/w of the composition. In some embodiments, the 13-methyltetradecanoate is present in an amount of about 1% to about 10% w/w, about 5% to about 15%, about 1% to about 5%, about 5% to about 10% w/w, or about 10% to about 15% w/w of the composition. In some embodiments, the 13-methyltetradecanoate is present in an amount of about 1% to about 5% w/w of the composition. In some embodiments, the 13-methyltetradecanoate is present in an amount of about 0.01% to about 0.3%, about 0.1% to about 0.3%, about 0.2% to about 0.5%, about 0.3% to about 0.5%, about 0.4% to about 0.6%, about 0.5% to about 0.7%, about 0.6% to about 0.8%, about 0.7% to about 0.9%, about 0.8% to about 1%, about 0.9% to about 1.1%, about 1% to about 1.2%, about 1.1% to about 1.3%, about 1.2% to about 1.4%, about 1.3% to about 1.5%, about 1.4% to about 1.6%, about 1.5% to about 1.7%, about 1.6% to about 1.8%, about 1.7% to about 1.9%, about 1.8% to about 2%, about 2% to about 2.5%, about 2.5% to about 3%, about 3% to about 3.5%, or about 3.5% to about 4% w/w of the composition. In some embodiments, the 13-methyltetradecanoate is present in an amount of about 0.01%, about 0.1%, about 0.2%, about 0.3%, about 0.4%, about 0.5%, about 0.6%, about 0.7%, about 0.8%, about 0.9%, about 1%, about 1.2%, about 1.4%, about 1.6%, about 1.8%, about 2%, about 2.2%, about 2.4%, about 2.6%, about 2.8%, about 3%, about 3.5%, about 4%, about 5%, about 6%, about 7%, about 8%, about 9%, about 10%, about 11%, about 12%, about 13%, about 14%, about 15%, about 16%, about 17%, about 18%, about 19%, or about 20% w/w of the composition.

As used herein, a “phytochemical” is a compound of plant origin that has antimicrobial properties. For example, a phytochemical can a compound that has antimicrobial properties and is produced by a plant through primary or secondary metabolism processes. In some embodiments, a phytochemical can be a polyphenol, an alkaloid, and/or a terpene or terpenoid. Non-limiting examples of a phytochemical include farnesol, caffeine, chlorogenic acid, a derivative of chlorogenic acid (e.g., (1S,3R,4R,5R)-3-(1-carboxy-2-(3,4-dihydroxyphenyl)ethoxy)-1,4,5-trihydroxycyclohexane-1-carboxylic acid), tannic acid, trigonolline, a hydroxycinnamic acid, quinic acid, gallic acid, and protocatechuic acid. Non-limiting examples of a hydroxycinnamic acid include caffeic acid, ferulic acid, and p-coumaric acid.

In some embodiments, a phytochemical as described herein has antimicrobial activity against a skin microorganism, i.e., a microorganism found on the skin of a subject, for example, a human. Many skin microorganisms are known to one of skill in the art, see, e.g., Byrd et al. Nat. Rev. Microbiol. 2018; 16(3):143-155, which is incorporated by reference herein in its entirety. In some embodiments, a phytochemical as described herein has antimicrobial activity against a skin microorganism that causes body odor. Non-limiting examples of a skin microorganism known to cause body odor include bacterial species from bacterial genuses including Corynebacterium, Micrococcus, Staphylococcus, Propionibacterium, Cutibacterium, Acinetobacter, Malassezia, and Brevibacterium. In some embodiments, a skin microorganism that causes body odor can include Staphylococcus hominis, Staphylococcus epidermidis, and Corynebacterium ssp. In some embodiments, the microorganism is a gram-positive bacterium.

Furthermore, compounds, e.g., compounds of plant origin, can be assayed for antimicrobial properties. For example, the activity of compounds of interest and/or plant origin can be assayed by growing the microorganisms of interest in media either containing or lacking the compound. The activity of the compounds of interest against microorganisms can be demonstrated by the ability of the compound to inhibit growth of defined strains of skin microorganisms. For this purpose, a panel of microorganisms can be assembled to include a variety of target skin-microorganism species. As another example, antimicrobial testing is typically performed to determine the minimum inhibitory concentration (MIC). Minimum inhibitory concentrations (MICs) can be determined by the microdilution method in a final volume of 100 μL according to protocols outlined by The Clinical and Laboratory Standards Institute (CLSI). Performance standards for reference strains are assessed within the same experimental design to maintain quality control. See, for example, Clinical Laboratory Standards Institute: Methods for dilution antimicrobial susceptibility tests for bacteria that grow aerobically M7-A8. Approved Standard-Eighth Edition. Wayne, PA: CLSI; December 2008; and Clinical Laboratory Standards Institute: Performance Standards for Antimicrobial Susceptibility Testing M100-S20; Approved Standard-Twentieth Edition. Wayne, PA: CLSI; June 2010. Further examples of methods to determine antimicrobial activity include an agar-dilution MIC assay and a time-kill kinetic assay, e.g., as described by Clinical Laboratory Standards Institute. For further examples, see Kepa et al. Biomed. Res. Int. 2018; 2018:7413504; Almeida et al. J. Agric. Food Chem. 2006; 54(23):8738-43; and Gohari et al. Daru. 2010; 18(1): 69-73; all of which are incorporated by reference herein in their entireties.

In some embodiments, a phytochemical exhibits antimicrobial activity against one or more bacterial species from bacterial genuses selected from the group consisting of: Corynebacterium, Micrococcus, Staphylococcus, Propionibacterium, Cutibacterium, Acinetobacter, Malassezia and Brevibacterium. In some embodiments, a phytochemical exhibits antimicrobial activity against one or more bacterial species selected from the group consisting of: Staphylococcus hominis, Staphylococcus epidermidis, and Corynebacterium ssp. In some embodiments, the phytochemical exhibits antimicrobial activity against a gram-positive bacterium. In some embodiments, the phytochemical exhibits antimicrobial activity against a skin microorganism known to cause body odor. In some embodiments, a phytochemical includes a compound extracted from a plant such as Camellia sinensis and/or a plant species from the genus Coffea. Non-limiting examples of a phytochemical extracted from Camellia sinensis and/or a plant species from the genus Coffea include farnesol, caffeine, caffeic acid, chlorogenic acid, a derivative of chlorogenic acid (e.g., (1S,3R,4R,5R)-3-(1-carboxy-2-(3,4-dihydroxyphenyl)ethoxy)-1,4,5-trihydroxycyclohexane-1-carboxylic acid), tannic acid, trigonolline, and protocatechuic acid.

In some embodiments, the phytochemical is present in an amount of about 0.01% to about 10%. For example about 0.01% to about 0.1%, about 0.01% to about 0.5%, about 0.01% to about 1%, about 0.01% to about 2%, about 0.01% to about 3%, about 0.01% to about 4%, about 0.01% to about 5%, about 0.01% to about 6%, about 0.01% to about 7%, about 0.01% to about 8%, about 0.01% to about 9%, about 9% to about 10%, about 8% to about 10%, about 7% to about 10%, about 6% to about 10%, about 5% to about 10%, about 4% to about 10%, about 3% to about 10%, about 2% to about 10%, about 1% to about 10%, or about 0.1% to about 10% w/w of the composition. For example, about 0.25% to about 0.5%, about 0.25% to about 0.75%, about 0.25% to about 1%, about 0.25% to about 1.5%, about 0.25% to about 2%, about 0.25% to about 2.5%, about 0.25% to about 3%, about 0.25% to about 3.5%, about 0.25% to about 4%, about 0.25% to about 4.5%, or about 0.25% to about 5% w/w of the composition. In some embodiments, the phytochemical is present in an amount of about 0.05%, about 0.1%, about 0.25%, about 0.5%, about 0.75%, about 1%, about 1.25%, about 1.5%, about 1.75%, about 2%, about 2.25%, about 2.5%, about 2.75%, about 3%, about 3.25%, about 3.5%, about 3.75%, about 4%, about 4.25%, about 4.5%, about 4.75%, about 5%, about 5.25%, about 5.5%, about 5.75%, about 6%, about 6.5%, about 7%, about 7.5%, about 8%, about 8.5%, about 9%, about 9.5%, or about 10% w/w of the composition.

In some embodiments, a phytochemical is selected from the group consisting of: farnesol, caffeine, chlorogenic acid, a derivative of chlorogenic acid, tannic acid, trigonolline, protocatechuic acid, a hydroxycinnamic acid, quinic acid, gallic acid, and a combination thereof. In some embodiments, the hydroxycinnamic acid is selected from the group consisting of: caffeic acid, ferulic acid, p-coumaric acid, and a combination thereof. In some embodiments, the derivative of chlorogenic acid is (1S,3R,4R,5R)-3-(1-carboxy-2-(3,4-dihydroxyphenyl)ethoxy)-1,4,5-trihydroxycyclohexane-l-carboxylic acid. In some embodiments, the phytochemical is a compound extracted from Camellia sinensis and/or a plant species from the genus Coffea.

In some embodiments, the phytochemical comprises caffeine. In some embodiments, caffeine is present in an amount of about 0.05% to about 5% w/w of the composition. For example, about 0.05% to about 0.1%, about 0.05% to about 0.15%, about 0.05% to about 0.25%, about 0.05% to about 0.5%, about 0.05% to about 1%, about 0.05% to about 1.5%, about 0.05% to about 2%, about 0.05% to about 3%, about 0.05% to about 4%, about 4% to about 5%, about 3% to about 5%, about 2% to about 5%, about 1% to about 5%, or about 0.5% to about 5% w/w of the composition. In some embodiments, caffeine is present in an amount of about 0.1% to about 2% w/w of the composition. For example, about 0.05% to about 0.5%, about 0.25% to about 0.75%, about 0.25% to about 1%, about 0.25% to about 1.5%, about 0.5% to about 0.75%, about 0.75% to about 1%, about 0.25% to about 1%, about 0.5% to about 1%, about 0.75% to about 1%, about 0.25% to about 2%, about 0.5% to about 2%, about 0.75% to about 2%, about 0.75% to about 2%, about 1% to about 2%, or about 1.5% to about 2% w/w of the composition. In some embodiments, caffeine is present in an amount of about 0.05%, about 0.1%, about 0.15%, about 0.2%, about 0.25%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, or about 2% w/w of the composition. In some embodiments, the phytochemical is caffeine.

In some embodiments, the phytochemical comprises farnesol. In some embodiments, farnesol is present in an amount of about 0.00001% to about 5% w/w of the composition. For example, about 0.00001% to about 0.00005%, about 0.00001% to about 0.0001%, about 0.00001% to about 0.0005%, about 0.00001% to about 0.001%, about 0.00001% to about 0.005%, about 0.00001% to about 0.01%, about 0.00001% to about 0.05%, about 0.00001% to about 0.1%, about 0.00001% to about 0.5%, about 0.00001% to about 1%, about 0.5% to about 1%, about 0.1% to about 1%, about 0.05% to about 1%, about 0.01% to about 1%, about 0.005% to about 1%, about 0.001% to about 1%, about 0.0005% to about 1%, about 0.0001% to about 1%, or about 0.00005% to about 1% w/w of the composition. For example, about 0.00001%, about 0.00005%, about 0.0001%, about 0.0005%, about 0.001%, about 0.005%, about 0.01%, or about 0.05% w/w of the composition. In some embodiments, farnesol is present in an amount of about 0.01% to about 0.05%, about 0.01% to about 0.1%, about 0.01% to about 0.5%, about 0.01% to about 1%, about 0.01% to about 1.5%, about 0.01% to about 2%, about 0.01% to about 3%, about 0.01% to about 4%, about 4% to about 5%, about 3% to about 5%, about 2% to about 5%, about 1% to about 5%, about 0.5% to about 5%, 0.1% to about 5%, or 0.05% to about 5% w/w of the composition. In some embodiments, farnesol is present in an amount of about 0.1% to about 0.5%, about 0.1% to about 0.75%, about 0.1% to about 1%, about 0.1% to about 1.5%, about 0.1% to about 2%, about 0.1% to about 2.5%, about 0.1% to about 3%, about 0.5% to about 0.75%, about 0.75% to about 1%, about 0.25% to about 1%, about 0.5% to about 1%, about 0.75% to about 1%, about 0.25% to about 2%, about 0.5% to about 2%, about 0.75% to about 2%, about 0.75% to about 2%, about 1% to about 2%, or about 1.5% to about 2% w/w of the composition. In some embodiments, farnesol is present in an amount of about 0.1%, about 0.25%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 2.2%, about 2.4%, about 2.6%, about 2.8%, about 3%, about 3.2%, about 3.4%, about 3.6%, about 3.8%, about 4%, about 4.2%, about 4.4%, about 4.6%, about 4.8%, or about 5% w/w of the composition. In some embodiments, the phytochemical is farnesol.

In some embodiments, the farnesol is present in the composition as a component of a plant extract or oil. As used herein, the term “plant extract” or “plant oil” refers to a mixture that is extracted from the tissue of a plant (e.g., a root, a stem, a trunk, a leaf, a seed, a fruit, a vegetable, and a flower). For example, a plant extract can be extracted from the tissue of a plant by treating the tissue with a solvent. As another example, a plant oil can be extracted by distillation, e.g., steam distillation. In some embodiments, the plant extract or oil can be a dermatologically acceptable plant extract or oil. In some embodiments, a “dermatologically acceptable plant extract” or a “dermatologically acceptable plant oil” refers to a plant extract or oil that is non-irritating and non-sensitizing. Non-limiting examples of a plant extract or oil that can contain farnesol include a plant extract or oil from a plant tissue from a plant genus such as Cymbopogon (e.g., Cymbopogon schoenanthus, Cymbopogon Flexuosus Oi, Cymbopogon nardus, Cymbopogon winterianus, Cymbopogon citratus, and Cymbopogon martinii); Prunus (e.g., Prunus armeniaca, Prunus brigantina, Prunus mandshurica, Prunus mume, Prunus zhengheensis and Prunus sibirica); Citrus (e.g., the flowers of Citrus aurantium (neroli) and a citrus peel); and Rosa (e.g., Rosa damascene). Further non-limiting examples of a plant extract or oil that can contain farnesol include a plant extract or oil from a plant tissue from Vachellia farnesiana, Matricaria chamomilla (chamomile); Abelmoschus moschatus (e.g., ambrette seeds); Myrocarpus fastigiatus (cabreuva); Oxystigma buccholtzii Harms; Cananga odorata (e.g., ylang-ylang); Acacia farnesiana; Myroxylon balsamum var. pereirae (e.g., Peru balsalm); Polianthes tuberosa (e.g., tuberose); Pimpinella anisum (e.g., anise seed), Aremisia campestris, Cyclamen Persicum (e.g., cyclamen), Myroxylon balsamum (e.g., tolu balsam), Cymbopogon nardus (e.g., ceylon citronella), Cymbopogon winterianus (java citronella), Cymbopogon martini (e.g., pamarosa), Cymbopogon schoenanthus (e.g., lemon grass), Cymbopogon Flexuosus Oi (e.g., lemon grass), Cymbopogon citratus (e.g., lemon grass), Prunus armeniaca (e.g., an apricot), Rosa damascene (rose), Citrus limon, Citrus medica, and Citrus aurantium (neroli).

In some embodiments, farnesol is present in the composition as a component of an extract or oil from a plant tissue from a plant genus selected from the group consisting of: Cymbopogon, Prunus, Citrus, Rosa, and a combination thereof. In some embodiments, farnesol is present in the composition as a component of an extract or oil from a plant tissue from a plant genus selected from the group consisting of: Cymbopogon, Prunus, Rosa, and a combination thereof. In some embodiments, farnesol is present in the composition as a component of an extract or oil from a plant selected from the group consisting of: Vachellia farnesiana, Matricaria chamomilla (chamomile); Abelmoschus moschatus (e.g., ambrette seeds); Myrocarpus fastigiatus (cabreuva); Oxystigma buccholtzii Harms; Cananga odorata (e.g., ylang-ylang); Acacia farnesiana; Myroxylon balsamum var. pereirae (e.g., Peru balsalm); Polianthes tuberosa (e.g., tuberose); Pimpinella anisum (e.g., anise seed), Aremisia campestris, Cyclamen Persicum (e.g., cyclamen), Myroxylon balsamum (e.g., tolu balsam), Cymbopogon nardus (e.g., ceylon citronella), Cymbopogon winterianus (java citronella), Cymbopogon martini (e.g., pamarosa), Cymbopogon schoenanthus (e.g., lemon grass), Cymbopogon Flexuosus Oi (e.g., lemon grass), Cymbopogon citratus (e.g., lemon grass), Prunus armeniaca (e.g., an apricot), Rosa damascene (rose), Citrus limon (e.g., Citrus limon (Lemon) fruit extract), Citrus medica, Citrus aurantium (neroli), and a combination thereof. In some embodiments, farnesol is present in the composition as a component of an extract or oil from a plant selected from the group consisting of: Vachellia farnesiana, Matricaria chamomilla (chamomile); Abelmoschus moschatus (e.g., ambrette seeds); Myrocarpus fastigiatus (cabreuva); Oxystigma buccholtzii Harms; Cananga odorata (e.g., ylang-ylang); Acacia farnesiana; Myroxylon balsamum var. pereirae (e.g., Peru balsalm); Polianthes tuberosa (e.g., tuberose); Pimpinella anisum (e.g., anise seed), Aremisia campestris, Cyclamen Persicum (e.g., cyclamen), Myroxylon balsamum (e.g., tolu balsam), Cymbopogon nardus (e.g., ceylon citronella), Cymbopogon winterianus (java citronella), Cymbopogon martini (e.g., pamarosa), Cymbopogon schoenanthus (e.g., lemon grass), Cymbopogon Flexuosus Oi (e.g., lemon grass), Cymbopogon citratus (e.g., lemon grass), Prunus armeniaca (e.g., an apricot), Rosa damascene (rose), and a combination thereof.

In some embodiments, the extract or oil from a plant tissue is present in the composition as a component of a lemon grass extract (e.g., an extract from Cymbopogon schoenanthus, Cymbopogon citratus, Cymbopogon Flexuosus, or a combination thereof). In some embodiments, the extract or oil from a plant tissue is present in an amount of about 0.01% to about 5% w/w of the composition. For example, about 0.01% to about 0.05%, about 0.01% to about 0.1%, about 0.01% to about 0.5%, about 0.01% to about 1%, about 0.01% to about 1.5%, about 0.01% to about 2%, about 0.01% to about 3%, about 0.01% to about 4%, about 4% to about 5%, about 3% to about 5%, about 2% to about 5%, about 1% to about 5%, about 0.5% to about 5%, 0.1% to about 5%, or 0.05% to about 5% w/w of the composition. In some embodiments, the extract or oil from a plant tissue extract is present in an amount of about 0.1% to about 0.5%, about 0.1% to about 0.75%, about 0.1% to about 1%, about 0.1% to about 1.5%, about 0.1% to about 2%, about 0.1% to about 2.5%, about 0.1% to about 3%, about 0.5% to about 0.75%, about 0.75% to about 1%, about 0.25% to about 1%, about 0.5% to about 1%, about 0.75% to about 1%, about 0.25% to about 2%, about 0.5% to about 2%, about 0.75% to about 2%, about 0.75% to about 2%, about 1% to about 2%, or about 1.5% to about 2% w/w of the composition. For example, about 0.1%, about 0.25%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 2.2%, about 2.4%, about 2.6%, about 2.8%, about 3%, about 3.2%, about 3.4%, about 3.6%, about 3.8%, about 4%, about 4.2%, about 4.4%, about 4.6%, about 4.8%, or about 5% w/w of the composition

In some embodiments, the farnesol is present in the composition as a component of a lemon grass extract (e.g., an extract from Cymbopogon schoenanthus, Cymbopogon citratus, Cymbopogon Flexuosus Oi, or a combination thereof). In some embodiments, the lemon grass extract is present in an amount of about 0.01% to about 5% w/w of the composition. For example, about 0.01% to about 0.05%, about 0.01% to about 0.1%, about 0.01% to about 0.5%, about 0.01% to about 1%, about 0.01% to about 1.5%, about 0.01% to about 2%, about 0.01% to about 3%, about 0.01% to about 4%, about 4% to about 5%, about 3% to about 5%, about 2% to about 5%, about 1% to about 5%, about 0.5% to about 5%, 0.1% to about 5%, or 0.05% to about 5% w/w of the composition. In some embodiments, the lemon grass extract is present in an amount of about 0.1% to about 0.5%, about 0.1% to about 0.75%, about 0.1% to about 1%, about 0.1% to about 1.5%, about 0.1% to about 2%, about 0.1% to about 2.5%, about 0.1% to about 3%, about 0.5% to about 0.75%, about 0.75% to about 1%, about 0.25% to about 1%, about 0.5% to about 1%, about 0.75% to about 1%, about 0.25% to about 2%, about 0.5% to about 2%, about 0.75% to about 2%, about 0.75% to about 2%, about 1% to about 2%, or about 1.5% to about 2% w/w of the composition. For example, about 0.1%, about 0.25%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 2.2%, about 2.4%, about 2.6%, about 2.8%, about 3%, about 3.2%, about 3.4%, about 3.6%, about 3.8%, about 4%, about 4.2%, about 4.4%, about 4.6%, about 4.8%, or about 5% w/w of the composition.

In some embodiments, the phytochemical is a combination of caffeine and farnesol.

In some embodiments, a composition as described herein includes an organic acid. Non-limiting examples of an organic acid include citric acid, malic acid, oxalic acid, manolic acid, malic acid, acetic acid, pyruvic acid, oxalic acid, glutaric acid, fumaric acid, formic acid, lactic acid, succinic acid, and α-ketoglutaric acid.

In some embodiments, the organic acid is present in an amount of about 0.01% to about 5% w/w of the composition. For example, about 0.01% to about 0.5%, about 0.01% to about 1%, about 0.01% to about 1.5%, about 0.01% to about 2%, about 0.01% to about 2.5%, about 0.01% to about 3%, about 0.01% to about 3.5%, about 0.01% to about 4%, or about 4% to about 5%, about 3.5% to about 5%, about 3% to about 5%, about 2.5% to about 5%, about 2% to about 5%, about 1.5% to about 5%, about 1% to about 5%, or about 0.5% to about 5% w/w of the composition. In some embodiments, the organic acid is present in an amount of about 0.1% to about 3%, about 0.1% to about 1%, about 0.1% to about 2%, about 0.5% to about 1.5%, about 1% to about 2%, about 1.5% to about 2.5%, or about 2% to about 3% w/w of the composition. For example, 0.1%, about 0.25%, about 0.5%, about 0.75%, about 1%, about 1.25%, about 1.5%, about 1.75%, about 2%, about 2.25%, about 2.5%, about 2.75%, or about 3% w/w of the composition.

In some embodiments, the organic acid is selected from the group consisting of: citric acid, malic acid, oxalic acid, manolic acid, malic acid, acetic acid, pyruvic acid, oxalic acid, glutaric acid, fumaric acid, formic acid, lactic acid, succinic acid, α-ketoglutaric acid, and a combination thereof.

In some embodiments, the organic acid is present in the composition as a component of an extract from a citrus fruit. Non-limiting examples of a citrus fruit extract include amanatsu (Citrus natsudaidai), balady citron (Citrus medica), bergamot orange (Citrus bergamia), bitter orange (Citrus x aurantium), blood orange (Citrus x sinensis), Buddha's hand (Citrus medica var. sarcodactylis), calamondin (Citrus mitis), Cam sành (Citrus reticulata x maxima), citron (Citrus medica), clementine (Citrus reticulate), Corsican citron (Citrus medica), desert lime (Citrus glauca), etrog (Citrus medica), finger lime (Citrus australasica), Florentine citron (Citrus x limonimedica), grapefruit (Citrus x paradise), greek citron (Citrus medica), hyaganatsu (Citrus tamurana), Anadomikan (Citrus x iyo), kabosu (Citrus sphaerocarpa), kaffir lime (Citrus hystrix), key lime (Citrus aurantiifolia), kinnow (Citrus nobilis x Citrus deliciosa), kiyomi (Citrus unshiu x Citrus sinensis), kumquat (Citrus japonica), lemon (Citrus limon), sweet lime (Citrus limetta), mandarin orange (Citrus reticulata), mangshanyegan (Citrus mangshanensis), meyer lemon (Citrus x meyeri), Moroccan citron (Citrus medica), chinotto (Citrus myrtifolia), orange (Citrus x sinensis), oroblanco (Citrus grandis x C. Paradisi/Citrus maxima/Citrus grandis), a papeda, Persian lime (Citrus x latifolia), pomelo (Citrus maxima or Citrus grandis), ponderosa lemon (Citrus maxima x medica), Rangpur (Citrus x limonia), round lime (Citrus australis), satsuma (Citrus unshiu), shangjuan (Citrus ichangensis x C. maxima), shonan gold (Citrus flaviculpus hort. ex Tanaka (Ōgonkan) x Citrus unshiu), sudachi (Citrus sudachi), Taiwan tangerine (Citrus x depressa), tangelo (C. reticulata x C. maxima or x C. paradise), tangerine (Citrus tangerine), tangor (C. reticulata x C. sinensis), ugli fruit (Citrus reticulata x Citrus paradise), and yuzu (Citrus ichangensis x C. reticulate).

In some embodiments, the extract from a citrus fruit is present in an amount of about 0.01% to about 5% w/w of the composition. For example, about 0.01% to about 0.5%, about 0.01% to about 1%, about 0.01% to about 1.5%, about 0.01% to about 2%, about 0.01% to about 2.5%, about 0.01% to about 3%, about 0.01% to about 3.5%, about 0.01% to about 4%, or about 4% to about 5%, about 3.5% to about 5%, about 3% to about 5%, about 2.5% to about 5%, about 2% to about 5%, about 1.5% to about 5%, about 1% to about 5%, or about 0.5% to about 5% w/w of the composition. In some embodiments, the extract from a citrus fruit is present in an amount of about 0.05% to about 2%, about 0.05% to about 1.5%, about 0.05% to about 1%, 0.05% to about 0.5%, about 0.5% to about 2%, about 1% to about 2%, or about 1.5% to about 2% w/w of the composition. In some embodiments, the extract from a citrus fruit is present in an amount of about 0.1% to about 3%, about 0.1% to about 1%, about 0.1% to about 2%, about 0.5% to about 1.5%, about 1% to about 2%, about 1.5% to about 2.5%, or about 2% to about 3% w/w of the composition. For example, about 0.05%, about 0.1%, about 0.25%, about 0.5%, about 0.75%, about 1%, about 1.25%, about 1.5%, about 1.75%, about 2%, about 2.25%, about 2.5%, about 2.75%, or about 3% w/w of the composition.

In some embodiments, the extract from a citrus fruit is selected from the group consisting of: amanatsu (Citrus natsudaidai), balady citron (Citrus medica), bergamot orange (Citrus bergamia), bitter orange (Citrus x aurantium), blood orange (Citrus x sinensis), Buddha's hand (Citrus medica var. sarcodactylis), calamondin (Citrus mitis), Cam sành (Citrus reticulata x maxima), citron (Citrus medica), clementine (Citrus reticulate), Corsican citron (Citrus medica), desert lime (Citrus glauca), etrog (Citrus medica), finger lime (Citrus australasica), Florentine citron (Citrus x limonimedica), grapefruit (Citrus x paradise), greek citron (Citrus medica), hyaganatsu (Citrus tamurana), Anadomikan (Citrus x iyo), kabosu (Citrus sphaerocarpa), kaffir lime (Citrus hystrix), key lime (Citrus aurantiifolia), kinnow (Citrus nobilis x Citrus deliciosa), kiyomi (Citrus unshiu x Citrus sinensis), kumquat (Citrus japonica), lemon (Citrus limon), sweet lime (Citrus limetta), mandarin orange (Citrus reticulata), mangshanyegan (Citrus mangshanensis), meyer lemon (Citrus x meyeri), Moroccan citron (Citrus medica), chinotto (Citrus myrtifolia), orange (Citrus x sinensis), oroblanco (Citrus grandis x C. Paradisi/Citrus maxima/Citrus grandis), a papeda, Persian lime (Citrus x latifolia), pomelo (Citrus maxima or Citrus grandis), ponderosa lemon (Citrus maxima x medica), Rangpur (Citrus x limonia), round lime (Citrus australis), satsuma (Citrus unshiu), shangjuan (Citrus ichangensis x C. maxima), shonan gold (Citrus flaviculpus hort. ex Tanaka (Ōgonkan) x Citrus unshiu), sudachi (Citrus sudachi), Taiwan tangerine (Citrus x depressa), tangelo (C. reticulata x C. maxima or x C. paradise), tangerine (Citrus tangerine), tangor (C. reticulata x C. sinensis), ugli fruit (Citrus reticulata x Citrus paradise), yuzu (Citrus ichangensis x C. reticulate), and a combination thereof.

In some embodiments, the extract from a citrus fruit is a citrus limon fruit extract. In some embodiments, the citrus limon fruit extract is present in an amount of about 0.01% to about 5% w/w of the composition. For example, about 0.01% to about 0.5%, about 0.01% to about 1%, about 0.01% to about 1.5%, about 0.01% to about 2%, about 0.01% to about 2.5%, about 0.01% to about 3%, about 0.01% to about 3.5%, about 0.01% to about 4%, or about 4% to about 5%, about 3.5% to about 5%, about 3% to about 5%, about 2.5% to about 5%, about 2% to about 5%, about 1.5% to about 5%, about 1% to about 5%, or about 0.5% to about 5% w/w of the composition. In some embodiments, the citrus limon fruit extract is present in an amount of about 0.1% to about 3%, about 0.1% to about 1%, about 0.1% to about 2%, about 0.5% to about 1.5%, about 1% to about 2%, about 1.5% to about 2.5%, or about 2% to about 3% w/w of the composition. For example, 0.1%, about 0.25%, about 0.5%, about 0.75%, about 1%, about 1.25%, about 1.5%, about 1.75%, about 2%, about 2.25%, about 2.5%, about 2.75%, or about 3% w/w of the composition.

In some embodiments, a composition as described herein includes antimicrobial peptide. Non-limiting examples of an antimicrobial peptide include lugdunin, human β-defensin-1 (hBD1), hBD2, hBD3, CAP18, a hepcidin, cathelicidin peptide LL-37, dermcidin (DCD-1), adrenomedullin, and elafin (SKALP) (see, e.g., Midorikawa et al. Infect. Immun. 2003; 71(7):3730-9; Agarwal et al. Med. Princ. Pract. 2016; 25(4):301-8; and Otto. Expert Rev. Dermatol. 2010; 5(2): 183-195; all of which are incorporated herein by reference in their entireties). In some embodiments, an antimicrobial peptide as described herein has antimicrobial activity against a skin microorganism, i.e., a microorganism found on the skin of a subject, for example, a human. In some embodiments, an antimicrobial peptide as described herein has antimicrobial activity against a skin microorganism that causes body odor. Furthermore, peptides can be assayed for antimicrobial properties using methods for screening for antimicrobial activity as described herein.

In some embodiments, the antimicrobial peptide is present in an amount of about 0.5 to about 25 pg/mL of the composition. For example, about 0.5 to about 1 pg/mL, about 0.5 to about 5 pg/mL, about 0.5 to about 10 pg/mL, about 0.5 to about 15 pg/mL, about 0.5 to about 20 pg/mL, about 20 to about 25 pg/mL, about 15 to about 25 pg/mL, about 10 to about 25 pg/mL, about 5 to about 25 pg/mL, or about 1 to about 25 pg/mL of the composition. In some embodiments, the antimicrobial peptide is present in an amount of about 1.5 to about 20.5 pg/mL, about 1 to about 10 pg/mL, about 5 to about 15 pg/mL, about 10 to about 20 pg/mL, or about 15 to about 25 pg/mL of the composition. For example, about 1 pg/mL, about 1.5 pg/mL, about 2 pg/mL, about 3 pg/mL, about 4 pg/mL, about 5 pg/mL, about 6 pg/mL, about 7 pg/mL, about 8 pg/mL, about 9 pg/mL, about 10 pg/mL, about 11 pg/mL, about 12 pg/mL, about 13 pg/mL, about 14 pg/mL, about 15 pg/mL, about 16 pg/mL, about 17 pg/mL, about 18 pg/mL, about 19 pg/mL, about 20 pg/mL, about 21 pg/mL, about 22 pg/mL, about 23 pg/mL, about 24 pg/mL, or about 25 pg/mL of the composition.

In some embodiments, the antimicrobial peptide is selected from the group consisting of: lugdunin, human β-defensin-1 (hBD1), hBD2, hBD3, CAP18, a hepcidin, cathelicidin peptide LL-37, dermcidin (DCD-1), adrenomedullin, elafin (SKALP), and a combination thereof. In some embodiments, the antimicrobial peptide exhibits antimicrobial activity against bacterial species from bacterial genuses selected from the group consisting of: Corynebacterium, Micrococcus, Staphylococcus, Propionibacterium, Brevibacterium, and a combination thereof. In some embodiments, the antimicrobial peptide exhibits antimicrobial activity against one or more of: Staphylococcus hominis, Staphylococcus epidermidis, and Corynebacterium ssp. In some embodiments, the antimicrobial peptide exhibits antimicrobial activity against a gram-positive bacterium. In some embodiments, the antimicrobial peptide exhibits antimicrobial activity against a skin microorganism known to cause body odor.

In some embodiments, the antimicrobial peptide is lugdunin. In some embodiments, lugdunin is present in an amount of about 0.5 to about 25 pg/mL of the composition. For example, about 0.5 to about 1 pg/mL, about 0.5 to about 5 pg/mL, about 0.5 to about 10 pg/mL, about 0.5 to about 15 pg/mL, about 0.5 to about 20 pg/mL, about 20 to about 25 pg/mL, about 15 to about 25 pg/mL, about 10 to about 25 pg/mL, about 5 to about 25 pg/mL, or about 1 to about 25 pg/mL of the composition. In some embodiments, lugdunin is present in an amount of about 1.5 to about 20.5 pg/mL, about 1 to about 10 pg/mL, about 5 to about 15 pg/mL, about 10 to about 20 pg/mL, or about 15 to about 25 pg/mL of the composition. For example, about 1 pg/mL, about 1.5 pg/mL, about 2 pg/mL, about 3 pg/mL, about 4 pg/mL, about 5 pg/mL, about 6 pg/mL, about 7 pg/mL, about 8 pg/mL, about 9 pg/mL, about 10 pg/mL, about 11 pg/mL, about 12 pg/mL, about 13 pg/mL, about 14 pg/mL, about 15 pg/mL, about 16 pg/mL, about 17 pg/mL, about 18 pg/mL, about 19 pg/mL, about 20 pg/mL, about 21 pg/mL, about 22 pg/mL, about 23 pg/mL, about 24 pg/mL, or about 25 pg/mL of the composition.

In some embodiments, a composition as described herein comprises: a yeast component (e.g., a yeast extract, yeast cell wall, or combination thereof); caffeine; and farnesol. In some embodiments, the composition further comprises a dermatologically acceptable carrier.

In some embodiments, a composition as described herein comprises:

A) one or more of:
a yeast component (e.g., a yeast extract, yeast cell wall, or combination thereof) present in an amount of at least about 0.5% w/w of the composition;
a Bacillaceae extract present in an amount of at least about 0.5% w/w of the composition; and
13-methyltetradecanoic acid present in an amount of at least about 0.5% w/w of the composition; and

B) one or more of:
a phytochemical present in an amount of at least about 0.01% to about 5% w/w of the composition; and
an organic acid present in an amount of at least about 0.01% to about 5% w/w of the composition.

In some embodiments, the phytochemical comprises farnesol. In some embodiments, the phytochemical comprises farnesol and caffeine. In some embodiments, the yeast component (e.g., a yeast extract, yeast cell wall, or combination thereof) is present in an amount of about 0.5% to about 10% w/w of the composition. In some embodiments, the Bacillaceae extract is present in an amount of about 0.5% to about 10% w/w of the composition, In some embodiments, the 13-methyltetradecanoic acid is present in an amount of at least about 0.5% to about 10% w/w of the composition. In some embodiments, the composition further comprises a dermatologically acceptable carrier.

In some embodiments, a composition as described herein comprises:

A) one or more of:
a yeast component (e.g., a yeast extract, yeast cell wall, or combination thereof) present in an amount of at least about 0.5% w/w of the composition;
a Bacillaceae extract present in an amount of at least about 0.5% w/w of the composition; and
13-methyltetradecanoic acid present in an amount of at least about 0.5% w/w of the composition; and

B) one or more of:
caffeine present in an amount of at least about 0.05% to about 5% w/w of the composition;
farnesol present in an amount of at least about 0.01% to about 5% w/w of the composition; and
an organic acid present in an amount of at least about 0.01% to about 5% w/w of the composition.

In some embodiments, the yeast component (e.g., a yeast extract, yeast cell wall, or combination thereof) is present in an amount of about 0.5% to about 10% w/w of the composition. In some embodiments, the Bacillaceae extract is present in an amount of about 0.5% to about 10% w/w of the composition, In some embodiments, the 13-methyltetradecanoic acid is present in an amount of at least about 0.5% to about 10% w/w of the composition. In some embodiments, the composition further comprises a dermatologically acceptable carrier.

In some embodiments, a composition as described herein comprises:

a yeast component (e.g., a yeast extract, yeast cell wall, or combination thereof) present in an amount of at least about 0.5% w/w of the composition;

farnesol present in an amount of at least about 0.01% to about 5% w/w of the composition; and

an organic acid present in an amount of at least about 0.01% to about 5% w/w of the composition. In some embodiments, the composition further comprises a dermatologically acceptable carrier.

In some embodiments, a composition as described herein comprises:

a yeast component (e.g., a yeast extract, yeast cell wall, or combination thereof) present in an amount of at least about 0.5% w/w of the composition;

caffeine present in an amount of at least about 0.05% to about 5% w/w of the composition; and

an organic acid present in an amount of at least about 0.01% to about 5% w/w of the composition. In some embodiments, the composition further comprises a dermatologically acceptable carrier.

In some embodiments, a composition as described herein comprises:

a yeast component (e.g., a yeast extract, yeast cell wall, or combination thereof) present in an amount of at least about 0.5% w/w of the composition;

caffeine present in an amount of at least about 0.05% to about 5% w/w of the composition; and

farnesol present in an amount of at least about 0.01% to about 5% w/w of the composition. In some embodiments, the composition further comprises a dermatologically acceptable carrier.

In some embodiments, a composition as described herein comprises: a yeast component (e.g., a yeast extract, yeast cell wall, or combination thereof); caffeine; farnesol; and an organic acid. In some embodiments, the composition further comprises a dermatologically acceptable carrier.

In some embodiments, a composition as described herein comprises:

a yeast component (e.g., a yeast extract, yeast cell wall, or combination thereof) present in an amount of at least about 0.5% w/w of the composition;

caffeine present in an amount of at least about 0.05% to about 5% w/w of the composition;

farnesol present in an amount of at least about 0.01% to about 5% w/w of the composition; and

an organic acid present in an amount of at least about 0.01% to about 5% w/w of the composition. In some embodiments, the composition further comprises a dermatologically acceptable carrier.

In some embodiments, a composition as described herein comprises: a yeast component (e.g., a yeast extract, yeast cell wall, or combination thereof); caffeine; farnesol; an organic acid; and lugdunin. In some embodiments, the composition further comprises a dermatologically acceptable carrier.

In some embodiments, a composition as described herein comprises:

a yeast component (e.g., a yeast extract, yeast cell wall, or combination thereof) present in an amount of at least about 0.5% w/w of the composition;

caffeine present in an amount of at least about 0.05% to about 5% w/w of the composition;

farnesol present in an amount of at least about 0.01% to about 5% w/w of the composition;

an organic acid present in an amount of at least about 0.01% to about 5% w/w of the composition; and

lugdunin present in an amount of about 1.5 to about 20.5 pg/mL of the composition. In some embodiments, the composition further comprises a dermatologically acceptable carrier.

In some embodiments, a composition as described herein can further include an antiperspirant. Non-limiting examples of an antiperspirant include: aluminum chloride, aluminum chlorohydrate, aluminum chlorohydrex polyethylene glycol complex, aluminum chlorohydrex propylene glycol complex, aluminum dichlorohydrate, aluminum dichlorohydrex polyethylene glycol complex, aluminum dichlorohydrex propylene glycol complex, aluminum sesquichlorohydrate, aluminum sesquichlorohydrex polyethylene glycol complex, aluminum sesquichlorohydrex propylene glycol complex, aluminum sulfate buffered with sodium aluminum lactate, aluminum zirconium octachlorohydrate, aluminum zirconium octachlorohydrex glycine complex, aluminum zirconium pentachlorohydrate, aluminum zirconium pentachlorohydrex glycine complex, aluminum zirconium tetrachlorohydrate, aluminum zirconium tetrachlorohydrex glycine complex, aluminum zirconium trichlorohydrate, and aluminum zirconium trichlorohydrex glycine complex.

In some embodiments, the composition further comprises an antiperspirant present in an amount of about 0.5% to about 25% w/w of the composition. For example, about 0.5% to 5%, about 0.5% to about 10%, about 0.5% to about 15%, about 0.5% to about 20%, about 20% to about 25%, about 15% to about 25%, about 10% to about 25%, or about 5% to about 25% w/w of the composition.

As used herein, the term “dermatologically acceptable carrier” refers to an agent that is useful in preparing topical solid, semi-solid, or liquid formulations. Non-limiting examples of a dermatologically acceptable carrier include a humectant, a liquid or solid emollient, a solubilizer and/or emulsifier, a preservative, a matrix agent, and the like. Further non-limiting examples of a dermatologically acceptable carrier include a Maranta Arundinacea (arrowroot) root powder a wax (e.g., ozokerite (earth wax), carnauba wax, candelilla wax, beeswax), tapioca starch, cornstarch, water, an alcohol (e.g., ethanol), glycerin, sodium stearate, diatomaceous earth, polyglyceryl-6 caprylate, polyglyceryl-4 caprate, triethyl citrate, Aloe barbadensis leaf juice, leuconostoc/radish root ferment filtrate, jojoba esters, stearyl alcohol, cellulose, silica, hydrated silica, sodium hydroxide, propanediol, leuconostoc/radish root ferment, stearic acid, ethylhexylglycerin, triethyl citrate, and titanium dioxide

In some embodiments, a dermatologically acceptable carrier comprises one or more of a humectant, an emollient, a solubilizer and/or emulsifier, a preservative, and a matrix agent. In some embodiments, a dermatologically acceptable carrier is present in an amount of about 0.05% to about 95% w/w of the composition. In some embodiments, a dermatologically acceptable carrier is present in an amount of about 0.05% to about 90% w/w of the composition. For example, about 0.05% to about 5%, about 0.05% to about 10%, about 0.05% to about 15%, about 0.05% to about 20%, about 0.05% to about 25%, about 0.05% to about 30%, about 0.05% to about 35%, about 0.05% to about 40%, about 0.05% to about 45%, about 0.05% to about 50%, about 0.05% to about 55%, about 0.05% to about 60%, about 0.05% to about 65%, about 0.05% to about 70%, about 0.05% to about 75%, about 0.05% to about 80%, about 0.05% to about 85%, about 85% to about 90%, about 80% to about 90%, about 75% to about 90%, about 70% to about 90%, about 65% to about 90%, about 60% to about 90%, about 55% to about 90%, about 50% to about 90%, about 45% to about 90%, about 40% to about 90%, about 35% to about 90%, about 30% to about 90%, about 25% to about 90%, about 20% to about 90%, about 15% to about 90%, about 10% to about 90%, or about 5% to about 90% w/w of the composition. In some embodiments, a dermatologically acceptable carrier is present in an amount of about 5% to about 10%, about 10% to about 20%, about 15% to about 25%, about 20% to about 30%, about 25% to about 35%, about 30% to about 40%, about 35% to about 45%, about 40% to about 50%, about 45% to about 55%, about 50% to about 60%, about 55% to about 65%, about 60% to about 70%, about 65% to about 75%, about 70% to about 80%, about 75% to about 85%, about 80% to about 90%, or about 85% to about 95% w/w of the composition.

In some embodiments, a dermatologically acceptable carrier comprises a humectant. A “humectant” as used herein refers to a substance used to reduce the loss of moisture that attracts water. For example, a humectant may attract water to bring moisture to the skin. Non-limiting examples of a humectant include erythritol, pentylene glycol, propanediol, sodium pyrrolidone carboxylic acid (PCA), sodium hyaluronate, betaine, glycerin, propylene glycol, a polyethylene glycol, a sugar, hexylene glycol, butylene glycol, Aloe vera gel, an alpha hydroxy acid such as lactic acid, glyceryl triacetate, lithium chloride, sorbitol, xylitol, maltitol, hyaluronic acid, allantoin, urea, tremella extract, dicyanamide, sodium lactate, Aloe barbadensis leaf juice, sodium L-pyroglutamate urea, and pyrrolidone carboxylic acid.

In some embodiments, the humectant is present in an amount of about 0.1% to about 50% w/w of the composition. For example, about 0.1% to about 5%, about 0.1% to about 10%, about 0.1% to about 15%, about 0.1% to about 20%, about 0.1% to about 25%, about 0.1% to about 30%, about 0.1% to about 35%, about 0.1% to about 40%, about 0.1% to about 45%, about 45% to about 50%, about 40% to about 50%, about 35% to about 50%, about 30% to about 50%, about 25% to about 50%, about 20% to about 50%, about 15% to about 50%, about 10% to about 50%, or about 5% to about 50% w/w of the composition. In some embodiments, the humectant is present in an amount of about 0.5% to about 5%, about 1% to about 10%, about 5% to about 15%, about 10% to about 20%, about 15% to about 25%, about 20% to about 30%, or about 25% to about 35% w/w of the composition. In some embodiments, the humectant is present in an amount of about 0.5%, about 1%, about 1.5%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30% w/w of the composition.

In some embodiments, the humectant comprises glycerin. In some embodiments, the humectant comprises a combination of glycerin and Aloe barbadensis leaf juice. In some embodiments, each humectant is independently present in an amount of about 0.1% to about 35% w/w of the composition. For example, about 0.1% to about 5%, about 0.1% to about 10%, about 0.1% to about 15%, about 0.1% to about 20%, about 0.1% to about 25%, about 0.1% to about 30%, about 30% to about 50%, about 25% to about 50%, about 20% to about 30%, about 15% to about 30%, about 10% to about 30%, or about 5% to about 30% w/w of the composition. In some embodiments, each humectant is independently present in an amount of about 0.5% to about 5%, about 1% to about 10%, about 5% to about 15%, about 10% to about 20%, about 15% to about 25%, or about 20% to about 30% w/w of the composition. In some embodiments, each humectant is independently present in an amount of about 0.5%, about 1%, about 1.5%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30% w/w of the composition.

In some embodiments, a dermatologically acceptable carrier comprises an emollient. An “emollient” as used herein refers to an agent that softens or soothes the skin by filling spaces between skin flakes and creating a smooth skin surface. Non-limiting examples of an emollient include an alkyl alcohol, a glycol alkyl ether, a short chain hydrocarbon, an oil, natural fatty acids, and an alkyl ester. Other non-limiting examples of an emollient include elastin, cetyl alcohol, a silicone, a coconut alkane, coco-caprylate/caprate, diheptyl succinate, capryloyl glycerin/sebacic acid copolymer, triethyl citrate, caprylic/capric triglyceride, a butter (e.g., Butyrospermum parkii butter (shea butter) or Theobroma cacao (cocoa) seed butter), collagen, colloidal oatmeal, elastin, glyceryl stearate, isopropyl palmitate, shea butter, coconut oil (Cocos nucifera oil), jojoba ester, stearyl alcohol, and stearic acid.

In some embodiments, an oil that can be used as an emollient in the compositions described herein includes a plant oil (e.g., a dermatologically acceptable plant oil). Many plant oils are named by the plant from which the oil is found. For example, rose oil or peppermint oil are derived from rose or peppermint plants, respectively. Plant oils can be extracted by several method known to those of skill in the art (e.g., steam distilled, enfleurage (i.e., extraction by using fat), maceration, solvent extraction, or mechanical pressing). Plant oils are insoluble in water and are soluble in alcohol, ether, fixed oils (vegetal), and other organic solvents. Typical physical characteristics found in plant oils include boiling points that vary from about 160° to 240° C. and densities ranging from about 0.759 to about 1.096.

In some embodiments, plant oils that can be used in the compositions described herein include oils derived from herbs, flowers, trees, and other plants. Exemplary plant oils include, but are not limited to avocado oil, olive oil, sunflower (Helianthus annuus) seed oil, soybean oil, cottonseed oil, palm oil, palm kernel oil, linseed oil, almond oil, castor oil, corn oil, rapeseed oil, sesame oil, safflower oil, wheat germ oil, peach kernel oil, macadamia nut oil, apricot kernel oil, sasanqua oil, Perilla oil, peanut oil, tea seed oil, Torreya nucifera oil, rice-bran oil, jojoba oil, sea buckthorn oil, hemp oil, flaxseed oil, walnut oil, tea tree oil, evening primrose oil, rosemary oil, coriander oil, thyme oil, pimento berries oil, rose oil, anise oil, balsam oil, bergamot oil, rosewood oil, cedar oil, chamomile oil, sage oil, clary sage oil, clove oil, cypress oil, eucalyptus oil, fennel oil, sea fennel oil, frankincense oil, geranium oil, ginger oil, grapefruit oil, jasmine oil, juniper oil, lavender oil, lemon oil, lemongrass oil, lime oil, mandarin oil, marjoram oil, myrrh oil, neroli oil, orange oil, patchouli oil, pepper oil, black pepper oil, petitgrain oil, pine oil, rose otto oil, sandalwood oil, spearmint oil, spikenard oil, vetiver oil, wintergreen oil, ylang ylang, bruiti oil, bacaba oil, acai oil, ojon oil, andiroba oil, coconut oil (Cocos nucifera oil), and tucuma oil. Other plant oils known to those of skill in the art are also contemplated as being useful when formulated in the compositions described herein.

In some embodiments, the emollient is present in an amount of about 0.1% to about 50% w/w of the composition. For example, about 0.1% to about 5%, about 0.1% to about 10%, about 0.1% to about 15%, about 0.1% to about 20%, about 0.1% to about 25%, about 0.1% to about 30%, about 0.1% to about 35%, about 0.1% to about 40%, about 0.1% to about 45%, about 45% to about 50%, about 40% to about 50%, about 35% to about 50%, about 30% to about 50%, about 25% to about 50%, about 20% to about 50%, about 15% to about 50%, about 10% to about 50%, or about 5% to about 50% w/w of the composition. In some embodiments, the emollient is present in an amount of about 0.5% to about 5%, about 1% to about 10%, about 5% to about 15%, about 10% to about 20%, about 15% to about 25%, about 20% to about 30%, or about 25% to about 35% w/w of the composition. In some embodiments, the emollient is present in an amount of about 0.5%, about 1%, about 1.5%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 40%, about 41%, about 42%, about 43%, about 44%, about 45%, about 46%, about 47%, about 48%, about 49%, or about 50% w/w of the composition. In some embodiments, a dermatologically acceptable carrier comprises one or more solubilizers and/or emulsifiers. A “solubilizer” or “emulsifier” as referred to herein is an agent that increases the solubility of another and/or increases the dispersion of an insoluble material. Non-limiting examples of a solubilizer or emulsifier include polyglyceryl-6 caprylate and polyglyceryl-4 caprate.

In some embodiments, the solubilizer and/or emulsifier is present in an amount of about 0.05% to about 10% w/w of the composition. For example, about 0.05% to about 0.1%, about 0.05% to about 0.5%, about 0.05% to about 1%, about 0.05% to about 2%, about 0.05% to about 3%, about 0.05% to about 4%, about 0.05% to about 5%, about 0.05% to about 6%, about 0.05% to about 7%, about 0.05% to about 8%, about 0.05% to about 9%, about 9% to about 10%, about 8% to about 10%, about 7% to about 10%, about 6% to about 10%, about 5% to about 10%, about 4% to about 10%, about 3% to about 10%, about 2% to about 10%, about 1% to about 10%, about 0.5% to about 10%, or about 0.1% to about 10% w/w of the composition. In some embodiments, the solubilizer and/or emulsifier is present in an amount of about 1% to about 5% w/w of the composition. In some embodiments, the solubilizer and/or emulsifier is present in an amount of 0.1% to about 2%, 0.5% to about 2.5%, about 1% to about 3%, about 1.5% to about 3.5%, about 2% to about 4%, about 2.5% to about 4.5%, or about 3% to about 5% w/w of the composition. In some embodiments, the solubilizer and/or emulsifier is present in an amount of about 0.5% to about 1.5%, about 0.5% to about 1.0%, about 1.5% to about 2%, about 1% to about 2%, about 0.5% to about 2%, or about 0.8% to about 2% w/w of the composition. For example, about 0.05% to about 0.4%, about 0.05% to about 0.2%, about 0.2% to about 0.5%, about 0.15% to about 0.25%, or about 0.1% to about 0.3% w/w of the composition. In some embodiments, the solubilizer and/or emulsifier is present in an amount of about 0.01%, about 0.05%, about 0.1%, about 0.5%, about 1%, about 1.5%, about 2%, about 2.5%, about 3%, about 3.5%, about 4%, about 4.5%, about 5%, about 5.5%, or about 6% w/w of the composition.

In some embodiments, a dermatologically acceptable carrier comprises one or more preservatives. In some embodiments, the one or more preservatives are selected from the group consisting of: ascorbic acid, an ascorbate, a palmitate, citric acid, a benzoate, a benzoic acid, a propionate, propionic acid, a sorbate, sorbic acid, a salicylic acid, a salicylate, hexa-2,4-dienoic acid, a hexa-2,4-dienoate, formaldehyde, a formaldehyde releaser, formic acid and its salts, 3-acetyl-6-methylpyran-2,4-(3H)-dione and its salts, 3,3′-dibromo-4,4′-hexamethylenedioxydibenzamidine and its salts, thiomersal, phenylmercuric salts, undec-10-enoic acid and its salts, 1,3-bis (2-ethylhexyl) hexahydro-5-methyl-5-pyrimidine, 5-bromo-5 -nitro-1,3-dioxane, bronopol, 2,4-dichlorobenzyl alcohol, 1-(4-chlorophenyl)-3-(3,4-dichlorophenyl) urea, chlorocresol, chloroxylenol, 5-chloro-2-(2,4-dichlorophenoxy) phenol, N,N″-methylenebis[N′-[3-(hydroxymethyl)-2,5-dioxoimidazolidin-4-yl]urea], polyaminopropyl biguanide, methenamine, quaternium-15, climbazole, DMDM hydantoin, benzyl alcohol, 1-Hydroxy-4-methyl-6-(2,4,4-trimethylpentyl)-2-pyridon, piroctone olamine, bromochlorophene, o-cymen-5-ol, chlorophene, chloroacetaminde, methylchloroisothiazolinone, methylisothiazolinone, phenoxyisopropanol, chlorhexidine, chlorhexidine diacetate, chlorhexidine digluconate, chlorhexidine dihydrochloride, dimethyl oxazolidine, behentrimonium chloride, cetri-monium bromide, cetrimonium chloride, laurtrimonium bromide, laurtrimonium chloride, steartrimonium bromide, steartrimonium chloride, diazolidinyl urea, hexamidine, hexamidine diisethionate, hexamidine paraben, glutaral, 7-ethylbicyclooxazolidine, chlorphenesin, sodium hydroxymethylglycinate, silver chloride, benzethonium chloride, benzalkonium chloride, benza-lkonium bromide, benzalkonium saccharinate, benzylhemiformal, iodopropynyl butylcarbamate, biphenyl-2-ol and its salts, pyrithionine zinc, an erythorbate, a nitrite, ethylenediaminetetraacetic acid (EDTA), butylated hydroxyanisole (BHA), butylated hydroxytoluene (BHT), capryllic acid, dilauryl thiodipropionate, erythorbic acid, gum guaiac, methylparaben, a sulfite, a bisulfite, a metabisulfite, propyl gallatepy, propylparaben, stannous chloride, sulfur dioxide, thiodipropionic acid, an isothiazoline, a paraben, phenoxyethanol, ethylhexylglycerin, a glycols, caprylhydroxamic acid, caprylyl glycol, glyceryl capylate, sodium benzoate, potassium sorbate, 1,2-hexanediol, propanediol, leuconostoc/radish root ferment, and a tocopherol. In some embodiments, the preservative is propanediol and/or leuconostoc/radish root ferment.

In some embodiments, the preservative is present in an amount of about 0.05% to about 10% w/w of the composition. For example, about 0.05% to about 0.1%, about 0.05% to about 0.5%, about 0.05% to about 1%, about 0.05% to about 2%, about 0.05% to about 3%, about 0.05% to about 4%, about 0.05% to about 5%, about 0.05% to about 6%, about 0.05% to about 7%, about 0.05% to about 8%, about 0.05% to about 9%, about 9% to about 10%, about 8% to about 10%, about 7% to about 10%, about 6% to about 10%, about 5% to about 10%, about 4% to about 10%, about 3% to about 10%, about 2% to about 10%, about 1% to about 10%, about 0.5% to about 10%, or about 0.1% to about 10% w/w of the composition. In some embodiments, the preservative is present in an amount of about 1% to about 5% w/w of the composition. In some embodiments, the preservative is present in an amount of 0.1% to about 2%, 0.5% to about 2.5%, about 1% to about 3%, about 1.5% to about 3.5%, about 2% to about 4%, about 2.5% to about 4.5%, or about 3% to about 5% w/w of the composition. In some embodiments, the preservative is present in an amount of about 0.5% to about 1.5%, about 0.5% to about 1.0%, about 1.5% to about 2%, about 1% to about 2%, about 0.5% to about 2%, or about 0.8% to about 2% w/w of the composition. For example, about 0.05% to about 0.4%, about 0.05% to about 0.2%, about 0.2% to about 0.5%, about 0.15% to about 0.25%, or about 0.1% to about 0.3% w/w of the composition. In some embodiments, the preservative is present in an amount of about 0.01%, about 0.05%, about 0.1%, about 0.5%, about 1%, about 1.5%, about 2%, about 2.5%, about 3%, about 3.5%, about 4%, about 4.5%, about 5%, about 5.5%, or about 6% w/w of the composition.

In some embodiments, a dermatologically acceptable carrier comprises one or more matrix agents. A “matrix agent” as referred to herein includes an inactive ingredient and/or a vehicle, e.g., a medium for conveying the active ingredient(s). A matrix agent can include an inert and/or inactive agent such as a bulking agent, a viscosity modifier, and/or a solvent. In some embodiments, a matrix agent can absorb wetness. In some embodiments, a matrix agent can be an aesthetic modifier. Non-limiting examples of a matrix agent include: corn starch, sodium bicarbonate (baking soda), magnesium hydroxide, sodium hydroxide, Maranta Arundinacea (arrowroot) root powder, a wax (e.g., ozokerite (earth wax), carnauba wax, candelilla wax, beeswax, microcrystalline wax, and Oryza sativa (rice) bran wax), tapioca starch, cornstarch, propanediol, water, sodium stearate, diatomaceous earth, cellulose, silica, hydrated silica, and titanium dioxide.

In some embodiments, the one or more matrix agents are present in an amount of about 5% to about 95% w/w of the composition. For example, about 5% to about 10%, about 5% to about 15%, about 5% to about 20%, about 5% to about 25%, about 5% to about 30%, about 5% to about 35%, about 5% to about 40%, about 5% to about 45%, about 5% to about 50%, about 5% to about 55%, about 5% to about 60%, about 5% to about 65%, about 5% to about 70%, about 5% to about 75%, about 5% to about 80%, about 5% to about 85%, about 5% to about 90%, about 90% to about 95%, about 85% to about 95%, about 80% to about 95%, about 75% to about 95%, about 70% to about 95%, about 65% to about 95%, about 60% to about 95%, about 55% to about 95%, about 50% to about 95%, about 45% to about 95%, about 40% to about 95%, about 35% to about 95%, about 30% to about 95%, about 25% to about 95%, about 20% to about 95%, about 15% to about 95%, or about 10% to about 95% w/w of the composition. In some embodiments, a dermatologically acceptable carrier is present in an amount of about 30% to about 80% w/w of the composition. In some embodiments, a dermatologically acceptable carrier is present in an amount of about 5% to about 10%, about 10% to about 20%, about 15% to about 25%, about 20% to about 30%, about 25% to about 35%, about 30% to about 40%, about 35% to about 45%, about 40% to about 50%, about 45% to about 55%, about 50% to about 60%, about 55% to about 65%, about 60% to about 70%, about 65% to about 75%, about 70% to about 80%, about 75% to about 85%, or about 80% to about 90% w/w of the composition. In some embodiments, each matrix agent is independently present in an amount of about 0.05% to about 90% w/w of the composition. For example, about 0.05% to about 5%, about 0.05% to about 10%, about 0.05% to about 15%, about 0.05% to about 20%, about 0.05% to about 25%, about 0.05% to about 30%, about 0.05% to about 35%, about 0.05% to about 40%, about 0.05% to about 45%, about 0.05% to about 50%, about 0.05% to about 55%, about 0.05% to about 60%, about 0.05% to about 65%, about 0.05% to about 70%, about 0.05% to about 75%, about 0.05% to about 80%, about 0.05% to about 85%, about 85% to about 90%, about 80% to about 90%, about 75% to about 90%, about 70% to about 90%, about 65% to about 90%, about 60% to about 90%, about 55% to about 90%, about 50% to about 90%, about 45% to about 90%, about 40% to about 90%, about 35% to about 90%, about 30% to about 90%, about 25% to about 90%, about 20% to about 90%, about 15% to about 90%, about 10% to about 90%, or about 5% to about 90% w/w of the composition. In some embodiments, a dermatologically acceptable carrier is present in an amount of about 5% to about 10%, about 10% to about 20%, about 15% to about 25%, about 20% to about 30%, about 25% to about 35%, about 30% to about 40%, about 35% to about 45%, about 40% to about 50%, about 45% to about 55%, about 50% to about 60%, about 55% to about 65%, about 60% to about 70%, about 65% to about 75%, about 70% to about 80%, about 75% to about 85%, or about 80% to about 90% w/w of the composition.

In some embodiments, the dermatologically acceptable carrier comprises one or more of: propanediol, water, glycerin, stearic acid, sodium stearate, diatomaceous earth, Maranta arundinacea (arrowroot) root powder, polyglyceryl-6 caprylate, polyglyceryl-4 caprate, ethylhexylglycerin, triethyl citrate, Aloe barbadensis leaf juice, leuconostoc/radish root ferment filtrate, jojoba esters, stearyl alcohol, cellulose, ethylhexylglycerin, silica, hydrated silica, and titanium dioxide. In some embodiments, the dermatologically acceptable carrier comprises: propanediol, water, glycerin, sodium stearate, diatomaceous earth, polyglyceryl-6 caprylate, polyglyceryl-4 caprate, ethylhexylglycerin, triethyl citrate, Aloe barbadensis leaf juice, leuconostoc/radish root ferment filtrate, jojoba esters, stearyl alcohol, cellulose, silica, hydrated silica, and titanium dioxide.

In some embodiments, the dermatologically acceptable carrier comprises: propanediol, water, glycerin, stearic acid, and sodium hydroxide. In some embodiments, the dermatologically acceptable carrier comprises: propanediol, water, glycerin, stearic acid, sodium hydroxide, silica, and hydrated silica. In some embodiments, the dermatologically acceptable carrier further comprises one or more of: diatomaceous earth, Maranta arundinacea (arrowroot) root powder, polyglyceryl-6 caprylate, polyglyceryl-4 caprate, leuconostoc/radish root ferment filtrate, Aloe barbadensis leaf juice, jojoba esters, cellulose, ethylhexylglycerin, triethyl citrate, stearyl alcohol, and titanium dioxide. In some embodiments, the dermatologically acceptable carrier is selected from the group consisting of: propanediol, water, glycerin, sodium stearate, diatomaceous earth, polyglyceryl-6 caprylate, polyglyceryl-4 caprate, ethylhexylglycerin, triethyl citrate, Aloe barbadensis leaf juice, leuconostoc/radish root ferment filtrate, jojoba esters, stearyl alcohol, cellulose, silica, hydrated silica, titanium dioxide, and a combination thereof.

In some embodiments, a compositions as described herein further includes a fragrance. Non-limiting examples of fragrances include amylcinnamal, amylcinnamyl alcohol, anisyl alcohol, benzyl alcohol, benzyl benzoate, benzyl cinnamate, benzyl salicylate, cinnamyl alcohol, cinnamal, citral, citronellolm, coumarin, eugenol, geraniol, hexyl cinnamicaldehyde, hydroxy-citronellal, hydroxy-methylpentylcyclohexenecarboxaldehyde, isoeugenol, D-limonene, linalool, methyl heptin carbonate, 3-methyl-4-(2,6,6-tri-methyl-2-cyclohexen-1-yl)-3-buten-2-one, oak moss and treemoss extract, treemoss extract, and -(4-tert-Butylbenzyl) propionaldehyde.

In some embodiments, the fragrance is present in an amount of about 0.05% to about 10% w/w of the composition. For example, about 0.05% to about 0.1%, about 0.05% to about 0.5%, about 0.05% to about 1%, about 0.05% to about 2%, about 0.05% to about 3%, about 0.05% to about 4%, about 0.05% to about 5%, about 0.05% to about 6%, about 0.05% to about 7%, about 0.05% to about 8%, about 0.05% to about 9%, about 9% to about 10%, about 8% to about 10%, about 7% to about 10%, about 6% to about 10%, about 5% to about 10%, about 4% to about 10%, about 3% to about 10%, about 2% to about 10%, about 1% to about 10%, about 0.5% to about 10%, or about 0.1% to about 10% w/w of the composition. In some embodiments, the fragrance is present in an amount of about 1% to about 5% w/w of the composition. In some embodiments, the fragrance is present in an amount of 0.1% to about 2%, 0.5% to about 2.5%, about 1% to about 3%, about 1.5% to about 3.5%, about 2% to about 4%, about 2.5% to about 4.5%, or about 3% to about 5% w/w of the composition. In some embodiments, the fragrance is present in an amount of about 0.5% to about 1.5%, about 0.5% to about 1.0%, about 1.5% to about 2%, about 1% to about 2%, about 0.5% to about 2%, or about 0.8% to about 2% w/w of the composition. For example, about 0.05% to about 0.4%, about 0.05% to about 0.2%, about 0.2% to about 0.5%, about 0.15% to about 0.25%, or about 0.1% to about 0.3% w/w of the composition. In some embodiments, the fragrance is present in an amount of about 0.01%, about 0.05%, about 0.1%, about 0.5%, about 1%, about 1.5%, about 2%, about 2.5%, about 3%, about 3.5%, about 4%, about 4.5%, about 5%, about 5.5%, or about 6% w/w of the composition.

In some embodiments, the composition is formulated for topical application. In some embodiments, the composition is in the form of a solid, a semi-solid, a lotion, an emulsion, or a liquid (e.g., a spray or roll-on).

In some embodiments, a composition described herein comprises:

A) one or more of:
a yeast component present in an amount of about 0.1% to about 20% w/w of the composition;
a Bacillaceae extract present in an amount of about 0.1% to about 20% w/w of the composition; and
13-methyltetradecanoic acid present in an amount of about 0.1% to about 20% w/w of the composition;

B) one or more of:
a phytochemical;
an organic acid; and
an antimicrobial peptide; and

C) a dermatologically acceptable carrier.

In some embodiments, the dermatologically acceptable carrier comprises one or more of: a humectant, a liquid or solid emollient, a solubilizer and/or emulsifier, a preservative, and a matrix agent. In some embodiments, the matrix agent is present in an amount of about 50% to about 95% w/w of the composition. In some embodiments, the matrix agent is present in an amount of about 80% to about 95% w/w of the composition. In some embodiments, the matrix agent comprises water. In some embodiments, the humectant is present in an amount of about 0.01% to about 20% w/w of the composition. In some embodiments, the humectant is present in an amount of about 0.01% to about 5% w/w of the composition. In some embodiments, the humectant comprises glycerin. In some embodiments, the dermatologically acceptable carrier comprises propanediol. In some embodiments, the preservative is present in an amount of about 0.01% to about 5% w/w of the composition. In some embodiments, the composition further comprises a fragrance (e.g., any of the fragrances described herein).

In some embodiments, the phytochemical comprises caffeine, farnesol, or a combination thereof. In some embodiments, the caffeine is present in an amount of at least about 0.05% to about 5% w/w of the composition. In some embodiments, the farnesol is present in the composition as a component of an extract or oil from a plant tissue from a plant genus selected from the group consisting of: Cymbopogon, Prunus, Rosa, and a combination thereof. In some embodiments, the extract or oil comprising farnesol is present in an amount of at least about 0.01% to about 5% w/w of the composition. In some embodiments, the organic acid is present in an amount of at least about 0.01% to about 5% w/w of the composition. In some embodiments, the antimicrobial peptide (e.g., lugdunin) is present in an amount of about 1.5 to about 20.5 pg/mL of the composition.

In some embodiments, a composition described herein comprises:

A) one or more of:
a yeast component present in an amount of about 1% to about 10% w/w of the composition;
a Bacillaceae extract present in an amount of about 1% to about 10% w/w of the composition; and
13-methyltetradecanoic acid present in an amount of about 1% to about 10% w/w of the composition;

B) one or more of:
a phytochemical;
an organic acid; and
an antimicrobial peptide; and

C) a dermatologically acceptable carrier. In some embodiments, the dermatologically acceptable carrier comprises one or more of: a humectant, a liquid or solid emollient, a solubilizer and/or emulsifier, a preservative, and a matrix agent. In some embodiments, the matrix agent is present in an amount of about 50% to about 95% w/w of the composition. In some embodiments, the matrix agent is present in an amount of about 80% to about 95% w/w of the composition. In some embodiments, the matrix agent comprises water. In some embodiments, the humectant is present in an amount of about 0.01% to about 20% w/w of the composition. In some embodiments, the humectant is present in an amount of about 0.01% to about 5% w/w of the composition. In some embodiments, the humectant comprises glycerin. In some embodiments, the dermatologically acceptable carrier comprises propanediol. In some embodiments, the preservative is present in an amount of about 0.01% to about 5% w/w of the composition. In some embodiments, the composition further comprises a fragrance (e.g., any of the fragrances described herein).

In some embodiments, the phytochemical comprises caffeine, farnesol, or a combination thereof. In some embodiments, the caffeine is present in an amount of at least about 0.05% to about 5% w/w of the composition. In some embodiments, the farnesol is present in the composition as a component of an extract or oil from a plant tissue from a plant genus selected from the group consisting of: Cymbopogon, Prunus, Rosa, and a combination thereof. In some embodiments, the extract or oil comprising farnesol is present in an amount of at least about 0.01% to about 5% w/w of the composition. In some embodiments, the organic acid is present in an amount of at least about 0.01% to about 5% w/w of the composition. In some embodiments, the antimicrobial peptide (e.g., lugdunin) is present in an amount of about 1.5 to about 20.5 pg/mL of the composition.

In some embodiments, the composition is a liquid composition. In some embodiments, the composition is a spray.

In some embodiments, a composition described herein comprises:

A) one or more of:
a yeast component present in an amount of about 0.1% to about 20% w/w of the composition;
a Bacillaceae extract present in an amount of about 0.1% to about 20% w/w of the composition; and
13-methyltetradecanoic acid present in an amount of about 0.1% to about 20% w/w of the composition;

B) one or more of:
a phytochemical;
an organic acid; and
an antimicrobial peptide; and

C) a dermatologically acceptable carrier. In some embodiments, the dermatologically acceptable carrier comprises one or more of: a humectant, a liquid or solid emollient, a solubilizer and/or emulsifier, a preservative, and a matrix agent. In some embodiments, the matrix agent is present in an amount of about 30% to about 75% w/w of the composition. In some embodiments, the matrix agent is present in an amount of about 35% to about 50% w/w of the composition. In some embodiments, the matrix agent comprises beeswax, candelilla wax, cornstarch, silica, sodium bicarbonate, or a combination thereof. In some embodiments, the emollient comprises about 0.1% to about 90% w/w of the composition. In some embodiments, the emollient comprises about 30% to about 60% w/w of the composition. In some embodiments, the emollient comprises shea butter, cocoa butter, coconut oil, caprylic/capric triglyceride, or a combination thereof. In some embodiments, the composition further comprises a fragrance (e.g., any of the fragrances described herein).

In some embodiments, the phytochemical comprises caffeine, farnesol, or a combination thereof. In some embodiments, the caffeine is present in an amount of at least about 0.05% to about 5% w/w of the composition. In some embodiments, the farnesol is present in the composition as a component of an extract or oil from a plant tissue from a plant genus selected from the group consisting of: Cymbopogon, Prunus, Rosa, and a combination thereof. In some embodiments, the extract or oil comprising farnesol is present in an amount of at least about 0.01% to about 5% w/w of the composition. In some embodiments, the organic acid is present in an amount of at least about 0.01% to about 5% w/w of the composition. In some embodiments, the antimicrobial peptide (e.g., lugdunin) is present in an amount of about 1.5 to about 20.5 pg/mL of the composition.

In some embodiments, a composition described herein comprises:

A) one or more of:
a yeast component present in an amount of about 0.1% to about 10% w/w of the composition;
a Bacillaceae extract present in an amount of about 0.1% to about 10% w/w of the composition; and
13-methyltetradecanoic acid present in an amount of about 0.1% to about 10% w/w of the composition;

B) one or more of:
a phytochemical;
an organic acid; and
an antimicrobial peptide; and

C) a dermatologically acceptable carrier. In some embodiments, the dermatologically acceptable carrier comprises one or more of: a humectant, a liquid or solid emollient, a solubilizer and/or emulsifier, a preservative, and a matrix agent. In some embodiments, the matrix agent is present in an amount of about 30% to about 75% w/w of the composition. In some embodiments, the matrix agent is present in an amount of about 35% to about 50% w/w of the composition. In some embodiments, the matrix agent comprises beeswax, candelilla wax, cornstarch, silica, sodium bicarbonate, or a combination thereof. In some embodiments, the emollient comprises about 0.1% to about 90% w/w of the composition. In some embodiments, the emollient comprises about 30% to about 60% w/w of the composition. In some embodiments, the emollient comprises shea butter, cocoa butter, coconut oil, caprylic/capric triglyceride, or a combination thereof. In some embodiments, the composition further comprises a fragrance (e.g., any of the fragrances described herein).

In some embodiments, the phytochemical comprises caffeine, farnesol, or a combination thereof. In some embodiments, the caffeine is present in an amount of at least about 0.05% to about 5% w/w of the composition. In some embodiments, the farnesol is present in the composition as a component of an extract or oil from a plant tissue from a plant genus selected from the group consisting of: Cymbopogon, Prunus, Rosa, and a combination thereof. In some embodiments, the extract or oil comprising farnesol is present in an amount of at least about 0.01% to about 5% w/w of the composition. In some embodiments, the organic acid is present in an amount of at least about 0.01% to about 5% w/w of the composition. In some embodiments, the antimicrobial peptide (e.g., lugdunin) is present in an amount of about 1.5 to about 20.5 pg/mL of the composition.

### Methods

Also provided herein are methods for reducing body odor comprising applying a composition as described herein to a subject. For example, in some embodiments, a composition as described herein reduces the body odor of a subject, e.g., where applied to the subject, compared to the body odor of a subject with no composition as described herein applied.

Also provided herein are methods for reducing the production of one or more malodorous compounds by bacteria in sweat comprising applying a composition as described herein to a subject. For example, in some embodiments, a composition as described herein reduces the production of one or more malodorous compounds by bacteria in sweat of a subject, e.g., where applied to the subject, compared to the production of one or more malodorous compounds by bacteria in sweat of a subject with no composition as described herein applied. In some embodiments, the one or more malodorous compounds are selected from the group consisting of: a thioalcohol, acetic acid, isovaleric acid, 3-methyl-2-hexenoic acid, propionic acid, and 3-hydroxy-3-methylhexanoic acid. In some embodiments, the bacteria are from a genus selected from the group consisting of: Corynebacterium, Micrococcus, Staphylococcus, Propionibacterium, Brevibacterium, Cutibacterium, and a combination thereof. In some embodiments, the bacteria are from a species selected from the group consisting of: S. hominis, S. aureus, S. epidermidis, Cutibacterium avidum, and a combination thereof.

In some embodiments, a composition as described herein is applied to the subject on an area selected from the group consisting of: an underarm, the face, a heel of a foot, a hairline, an inner thigh, an area behind a knee, a sole of a foot, a hand, the groin such as genitalia and the perineum, and a combination thereof.

In some embodiments, a composition as described herein is applied daily. In some embodiments, a composition as described herein is applied about once a day, about once every two days, or about once every three days. In some embodiments, the composition is applied to the subject from about one to about six times daily, or from about one to about five times daily, or from about one to about four times daily, or from about one to about three times daily, or from about one to about two times daily.

In some embodiments, a composition as described herein is applied after bathing. In some embodiments, a compositions as described herein is applied after washing the application area, e.g., an underarm, the face, a heel of a foot, a hairline, an inner thigh, an area behind a knee, a sole of a foot, a hand, the groin such as genitalia and the perineum, and a combination thereof. In some embodiments, a compositions as described herein is applied at night. In some embodiments, a compositions as described herein is applied prior to going to sleep. In some embodiments, a compositions as described herein is applied in the morning.

### EXAMPLES

The invention will be described in greater detail by way of specific examples. The following examples are offered for illustrative purposes, and to exemplify the compositions and methods described herein and are not intended to limit the invention in any manner. Many variations will suggest themselves and are within the full intended scope. Those of skill in the art will readily recognize a variety of non-critical parameters that can be changed or modified to yield essentially the same results.

### Example 1

### Preparation of a Deodorant Composition

Propanediol, water, and 50% sodium hydroxide were added to a first tank. In a separate tank, glycerin and cellulose were mixed to form slurry with little to no lumps. The glycerin and cellulose mixture was then added to the first tank with the propanediol, water, and sodium hydroxide, and heating of the mixture to 85-90° C. was started. When the mixture was at 45-50° C., the Aloe vera, lemongrass extract, and lemon extract were added one at a time with mixing. When the mixture reached 85-90° C., stearic acid and stearyl alcohol were added with mixing. Stearic acid may make the mixture chunky and solidify, so proper mixing and recirculation is needed. The mixture was kept at 85-90° C. and mixed until all solids were dissolved.

In a separate tank, the TEGO® Solve 90, ethylhexylglycerin, and triethyl citrate were mixed together. This mixture was then added to the first tank and mixed until uniform while maintaining the temperature at 85-90° C. While maintaining the temperature at 85-90° C., DEOPLEX® (Saccharomyces Ferment), Yeast Essence E100 (Yeast Extract), and Leuconostoc/radish root ferment filtrate were added to the first tank one at a time with mixing until uniform.

The pH of the first tank was checked; the pH should be around a pH of 8-9. 50% sodium hydroxide was added to the first tank until the mixture reached a pH of 10-11 (about 0.02-0.05% of 50% sodium hydroxide). Mixing should be maintained to prevent solidification.

While maintaining the mixture at 85-90° C., the remaining ingredients are added to the first tank one at a time with mixing until uniform and homogenized until all powders are uniform. The mixture is then cooled to 75-80° C. The mixture is then filled into the packaging while maintaining the temperature at 75-80° C. and continuously mixing.

A composition as shown in Table 1 was prepared using the protocol described above.

TABLE 1
Deodorant composition

| | % w/w |
|---|---|
| PART A | |
| Propanediol (e.g., ZEMEA ®) | 36 |
| DI Water | 15.86 |
| Sodium Hydroxide (50% Solution) | 1.9 |
| PART B | |
| Glycerin (e.g., 99.5%, 99.7%) | 20 |
| Cellulose (e.g., SENSOCEL ® 90) | 1 |
| PART C | |
| Aloe Vera Extract in Glycerin | 0.01 |
| Lemongrass Extract in Glycerin | 1 |
| Lemon Extract in Glycerin | 0.1 |
| PART D | |
| Stearyl Alcohol | 0.3 |
| Stearic Acid Triple Press (RITASTEARIC ®) | 6 |
| PART E | |
| Polyglyceryl-6 Caprylate, Polyglyceryl-4 Caprate | 2.5 |
| (e.g., TEGOSOLVE ®-90) | |
| Ethylhexylglycerin (e.g., LEXGARD ® E) | 0.4 |
| Triethyl citrate | 0.4 |
| PART F | |
| Yeast Extract (e.g., Yeast Essence E100) | 5 |
| Leuconostoc/Radish Root Ferment Filtrate | 0.01 |
| (e.g., LEUCIDAL ® Liquid) | |
| Saccharomyces Ferment (e.g., DEOPLEX CLEAR ®) | 1.5 |
| PART G | |
| Sodium Hydroxide (50% Solution) | QS |
| PART H | |
| Hydrated Silica/Titanium Dioxide/Jojoba Esters | 0.6 |
| (e.g., A15-TiO2-S-NJE10) | |
| Silica (e.g., CAB-O-SIL ®) | 0.65 |
| Hydrated Silica (e.g., SYLOID ®) | 0.65 |
| Natural Anhydrous Caffeine | 0.1 |
| Maranta Arundinacea (Arrowroot) Root Powder | 3 |
| Diatomaceous Earth (IMERCARE ®) | 3 |

### Example 2

### Stability Testing of Deodorant Composition

To determine the quality and stability of the deodorant composition in Example 1 in various conditions, the deodorant composition was tested in glass jars in various conditions including freeze/thaw cycles, and at 5° C., room temperature (25° C.), 37° C., and 45° C. for 12 weeks. Observations were made a 2, 4, 8, and 12 weeks. The results for freeze/thaw cycles are in Table 2 and for stability at various temperatures in Table 3.

TABLE 2
Results of freeze/thaw stability tests.

| Condition: Freeze/Thaw | Observations |
|---|---|
| Cycle 1 | Very minimal sweating (droplets of |
| | moisture) apparent. 1-2 small droplets |
| | (less than 0.2 mm) |
| Cycle 2 | A few small (less than 0.2 mm) droplets |
| | of moisture apparent on surface. |
| | Reabsorbs within 1 hour. |
| Cycle 3 | A few small (less than 0.2 mm) droplets |
| | of moisture apparent on surface with |
| | some apparent liquid on sides of glass |
| | jar. Reabsorbs within 1 hour. |

TABLE 3
Results of temperature stability tests.

| Condition | Week 2 | Week 4 | Week 8 | Week 12 |
|---|---|---|---|---|
| 5° C. | COA* Good, | COA* Good, | COA* Good, | COA* Good, |
| | No change to | No change to | Sweating (A | Sweating (A |
| | initial | initial | few droplets) | few droplets) |
| | | | apparent on | apparent on |
| | | | surface and | surface and |
| | | | side of jar but | side of jar but |
| | | | reabsorbs after | reabsorbs after |
| | | | 24 hrs | 24 hrs. No |
| | | | | more sweating |
| | | | | observed than |
| | | | | Week 8 |
| 25° C. | COA* Good, | COA* Good, | COA* Good, | COA* Good, |
| | No change to | No change to | No change to | No change to |
| | initial | initial | initial | initial |
| 37° C. | COA* Good, | COA* Good, | COA* Good | COA* Good |
| | No change to | No change to | with a slightly | with a slightly |
| | initial | initial | stronger odor | stronger odor |
| | | | of yeast | of yeast. |
| | | | | Stronger odor |
| | | | | than Week 8 |
| 45° C. | COA* Good, | COA* Good, | COA* Good | COA* Good |
| | No change to | No change to | with a slightly | with a slightly |
| | initial | initial | stronger odor | stronger odor |
| | | | of yeast | of yeast. |
| | | | | Stronger odor |
| | | | | than Week 8. |
| | | | | Some droplets |
| | | | | of moisture |
| | | | | appeared in |
| | | | | glass jar but |
| | | | | none on |
| | | | | product. |

*COA: Color, Odor, Appearance

As shown in Table 2 and Table 3, the deodorant composition was stable under all tested conditions during the 12-week stability test.

### Example 3

### Water-Based Spray Deodorant Composition

A composition as shown in Table 4 is prepared. The ingredients in Part A of Table 4 are added to a tank and mixed until uniform. Once uniform, the ingredients from Part B are added and mixed until uniform. The pH is adjusted to 5-6.

TABLE 4
Water-based spray deodorant composition

| | % w/w |
|---|---|
| PART A | |
| DI Water | 86.89 |
| Propanediol (e.g., ZEMEA ®) | 1.5 |
| Ethylhexylglycerin (e.g., LEXGARD ® E) | 0.5 |
| Caprylhydroxamic Acid, Propanediol (e.g., ZEASTAT ®) | 2.5 |
| PART B | |
| Vegetable Protein Extract A3058 (e.g., DEOPLEX CLEAR ®) | 2.5 |
| Yeast Extract | 5 |
| Lemon grass Extract in Glycerin | 1 |
| Lemon Extract in Glycerin | 0.1 |
| 50% Citric Acid | QS |

### Example 4

### Anhydrous-Based Deodorant Composition

A composition as shown in Table 5 is prepared. The ingredients in Part A of Table 5 are added to a tank, which is heated to 80-85° C. and mixed. The mixture is mixed and held at 80-85° C. until uniform and all solids are melted. The mixture is then cooled to 70-75° C. Once 70-75° C. is reached, the ingredients from Part B of Table 5 are added. While maintaining the temperature at 70-75° C., the mixture is homogenized and mixed until uniform. Once uniform, homogenization is stopped and the mixture is cooled to 65-70° C. While maintaining the temperature at 65-70° C., the Part C ingredients are added with moderate mixing. Once Part C is fully incorporated, begin cooling the mixture to 55-60° C. with moderate mixing. At 55-60° C., pour the mixture into packaging and cool appropriately.

TABLE 5
Anhydrous-based deodorant composition.

| | % w/w |
|---|---|
| PART A | |
| Caprylic/Capric Triglyceride | 10.38 |
| Butyrospermum Parkii (Shea) Butter | 5.3 |
| Theobroma Cacao (Cocoa) Seed Butter (Cocoa Butter) | 19.75 |
| Coconut Oil | 9 |
| Beeswax | 11 |
| Euphorbia Cerifera (Candelilla) Wax | 7 |
| Lemon Fruit Extract in Sunflower Oil | 0.5 |
| Capryloyl Glycerin/Sebacic Acid Copolymer | 12.24 |
| (e.g. LEXFEEL ® N100 MB) | |
| PART B | |
| Sodium Bicarbonate | 4 |
| Zea Mays (Corn) Starch (e.g., MAISITA ® 21.050) | 15 |
| Yeast Extract | 0.25 |
| Silica (MSS-500W) | 5.56 |
| PART C | |
| Vegetable Protein Extract A3058 (e.g., DEOPLEX CLEAR ®) | 0.01 |

### Example 5

### Anhydrous-Based Deodorant Composition

A composition as shown in Table 6 is prepared. The ingredients in Part A of Table 6 are added to a tank, which is heated to 80-85° C. and mixed. The mixture is mixed and held at 80-85° C. until uniform and all solids are melted. The mixture is then cooled to 70-75° C. Once 70-75° C. is reached, the ingredients from Part B of Table 6 are added. While maintaining the temperature at 70-75° C., the mixture is homogenized and mixed until uniform. Once uniform, homogenization is stopped and the mixture is cooled to 58-62° C. At 58-62° C., pour the mixture into packaging and cool appropriately

TABLE 6
Anhydrous-based deodorant composition.

| | % w/w |
|---|---|
| PART A | |
| Coconut Oil | 25.8 |
| Sunflower Oil | 30 |
| Carnauba Wax #1 | 3.25 |
| Candelilla Wax Yellow | 14.75 |
| Shea Butter | 3 |
| Jojoba Esters (e.g., FLORAESTERS ® 60) | 0.9 |
| Tocopherol | 0.3 |
| PART B | |
| Arrowroot Powder | 10 |
| Sodium Bicarbonate USP #1 | 10 |
| Vegetable Protein Extract A3058 (e.g., DEOPLEX CLEAR ®) | 0.001 |

### Other Embodiments

1. A dermatologically acceptable composition comprising:
   A) one or more of:
      a yeast component;
      a Bacillaceae extract; and
      13-methyltetradecanoic acid;
   B) one or more of:
      a phytochemical;
      an organic acid; and
      an antimicrobial peptide; and
   C) a dermatologically acceptable carrier.
2. A dermatologically acceptable composition comprising:
   A) a yeast component, a Bacillaceae extract, or a combination thereof;
   B) one or more of:
      a phytochemical;
      an organic acid; and
      an antimicrobial peptide; and
   C) a dermatologically acceptable carrier.
3. A dermatologically acceptable composition comprising:
   A) a yeast component, 13-methyltetradecanoic acid, or a combination thereof;
   B) one or more of:
      a phytochemical;
      an organic acid; and
      an antimicrobial peptide; and
   C) a dermatologically acceptable carrier.
4. A dermatologically acceptable composition comprising:
   A) a Bacillaceae extract, 13-methyltetradecanoic acid, or a combination thereof;
   B) one or more of:
      a phytochemical;
      an organic acid; and
      an antimicrobial peptide; and
   C) a dermatologically acceptable carrier.
5. A dermatologically acceptable composition comprising:
   a yeast component;
   a phytochemical;
   an organic acid;
   and a dermatologically acceptable carrier.
6. A dermatologically acceptable composition comprising:
   a yeast component;
   a phytochemical; and
   a dermatologically acceptable carrier.
7. A dermatologically acceptable composition comprising:
   a Bacillaceae extract, 13-methyltetradecanoic acid, or a combination thereof;
   a phytochemical; and
   a dermatologically acceptable carrier.
8. The dermatologically acceptable composition of any one of embodiments 1-3 and 4-6, wherein the yeast component is a yeast extract a yeast cell wall, or a combination thereof.
9. The dermatologically acceptable composition of any one of embodiments 1-3, 4-6, and 8, wherein the yeast component is present in an amount of at least 0.01% w/w of the composition.
10. The dermatologically acceptable composition of any one of embodiments 1-3, 4-6, and 8-9, wherein the yeast component is present in an amount of about 0.5% to about 20% w/w of the composition.
11. The dermatologically acceptable composition of any one of embodiments 1-3, 4-6, and 8-10, wherein the yeast component is present in an amount of at least 1% w/w of the composition.
12. The dermatologically acceptable composition of any one of embodiments 1-3, 4-6, and 8-11, wherein the yeast component is present in an amount of about 1% to about 10% w/w of the composition.
13. The dermatologically acceptable composition of any one of embodiments 1-3, 4-6, and 8-12, wherein the yeast component is present in an amount of about 4% to about 8% w/w of the composition.
14. The dermatologically acceptable composition of any one of embodiments 1-3, 4-6, and 8-13, wherein the yeast component is present in an amount of about 1% to about 5% w/w of the composition.
15. The dermatologically acceptable composition of any one of embodiments 1-3, 4-6, and 8-14, wherein the yeast extract comprises: a Saccharomyces extract, a Candida extract, a Debaryomyces extract, a Kloeckera extract, a Kluyveromyces extract, a Geotrichum extract, a Pichia extract, a Wickerhamomyces extract, or a combination thereof.
16. The dermatologically acceptable composition of any one of embodiments 1-3, 4-6, and 8-15, wherein the yeast extract comprises: Saccharomyces boulardii extract, Saccharomyces cerevisiae extract, Saccharomyces pastorianus extract. Candida bombicola extract, Debaryomyces hansenii extract, Kloeckera apiculate extract, Kluyveromyces thermotolerans extract, Geotrichum candidum extract, Candida intermedia extract, Kluyveromyces marxianus extract, Pichia norvegensis extract, Pichia fermentans extract, Candida tropicalis extract, Candida apicola extract, Wickerhamiella domercqiae extract, Wickerhamomyces anomalus extract, and a combination thereof.
17. The dermatologically acceptable composition of any one of embodiments 1-3, 4-6, and 8-16, wherein the yeast extract is Saccharomyces pastorianus extract.
18. The dermatologically acceptable composition of any one of embodiments 1-3, 4-6, and 8-17, wherein the yeast extract is Saccharomyces boulardii extract.
19. The dermatologically acceptable composition of any one of embodiments 1-3, 4-6, and 8-18, wherein the yeast extract is Saccharomyces cerevisiae extract.
20. The dermatologically acceptable composition of any one of embodiments 1-3, 4-6, and 8-19, wherein the yeast extract is a Saccharomyces boulardii and Saccharomyces cerevisiae extract.
21. The dermatologically acceptable composition of any one of embodiments 1-3, 4-6, and 8-20, wherein the yeast cell wall comprises: a Saccharomyces cell wall, a Candida cell wall, a Debaryomyces cell wall, a Kloeckera cell wall, a Kluyveromyces cell wall, a Geotrichum cell wall, a Pichia cell wall, a Wickerhamomyces cell wall, or a combination thereof.
22. The dermatologically acceptable composition of any one of embodiments 1-3, 4-6, and 8-21, wherein the yeast cell wall comprises: Saccharomyces boulardii cell wall, Saccharomyces cerevisiae cell wall, Saccharomyces pastorianus cell wall, Candida bombicola cell wall, Debaryomyces hansenii cell wall, Kloeckera apiculate cell wall, Kluyveromyces thermotolerans cell wall, Geotrichum candidum cell wall, Candida intermedia cell wall, Kluyveromyces marxianus cell wall, Pichia norvegensis cell wall, Pichia fermentans cell wall, Candida tropicalis cell wall, Candida apicola cell wall, Wickerhamiella domercqiae cell wall, Wickerhamomyces anomalus cell wall, and a combination thereof.
23. The dermatologically acceptable composition of any one of embodiments 1-3, 4-6, and 8-22, wherein the yeast cell wall is Saccharomyces pastorianus cell wall.
24. The dermatologically acceptable composition of any one of embodiments 1-3, 4-6, and 8-22, wherein the yeast cell wall is Saccharomyces boulardii cell wall.
25. The dermatologically acceptable composition of any one of embodiments 1-3, 4-6, and 8-22, wherein the yeast cell wall is Saccharomyces cerevisiae cell wall.
26. The dermatologically acceptable composition of any one of embodiments 1-3, 4-6, and 8-22, wherein the yeast cell wall is a Saccharomyces boulardii and Saccharomyces cerevisiae cell wall.
27. The dermatologically acceptable composition of any one of embodiments 1, 2, 4, and 7-26, wherein the Bacillaceae extract is present in an amount of about 0.1% to about 20% w/w of the composition.
28. The dermatologically acceptable composition of any one of embodiments 1, 2, 4, and 7-27, wherein the Bacillaceae extract is present in an amount of at least 1% w/w of the composition.
29. The dermatologically acceptable composition of any one of embodiments 1, 2, 4, and 7-28, wherein the Bacillaceae extract is present in an amount of about 1% to about 5% w/w of the composition.
30. The dermatologically acceptable composition of any one of embodiments 1, 2, 4, and 7-29, wherein the Bacillaceae extract is an Anoxybacillus extract.
31. The dermatologically acceptable composition of any one of embodiments 1, 2, 4, and 7-30, wherein the Bacillaceae extract is an Anoxybacillus kamchatkensis extract.
32. The dermatologically acceptable composition of any one of embodiments 1, 3, 4, and 7-31, wherein the 13-methyltetradecanoic acid is present in an amount of about 0.01% to about 20% w/w of the composition.
33. The dermatologically acceptable composition of any one of embodiments 1, 3, 4, and 7-32, wherein the 13-methyltetradecanoic acid is present in an amount of at least 0.1% w/w of the composition.
34. The dermatologically acceptable composition of any one of embodiments 1, 3, 4, and 7-33, wherein the 13-methyltetradecanoic acid is present in an amount of about 0.1% to about 5% w/w of the composition.
35. The dermatologically acceptable composition of any one of embodiments 1-34, wherein the phytochemical is present in an amount of about 0.1% to about 10% w/w of the composition.
36. The dermatologically acceptable composition of any one of embodiments 1-35, wherein the phytochemical is present in an amount of about 0.5% to about 6% w/w of the composition.
37. The dermatologically acceptable composition of any one of embodiments 1-36, wherein the phytochemical is present in an amount of about 0.5% to about 4% w/w of the composition.
38. The dermatologically acceptable composition of any one of embodiments 1-37, wherein the phytochemical is selected from the group consisting of: farnesol, caffeine, caffeic acid, chlorogenic acid, a derivative of chlorogenic acid, tannic acid, trigonolline, protocatechuic acid, and a combination thereof.
39. The dermatologically acceptable composition of any one of embodiments 1-38, wherein the phytochemical is a compound extracted from Camellia sinensis and/or a plant species from the genus Coffea.
40. The dermatologically acceptable composition of any one of embodiments 1-39, wherein the phytochemical comprises caffeine.
41. The dermatologically acceptable composition of embodiment 40, wherein caffeine is present in an amount of about 0.1% to about 2% w/w of the composition.
42. The dermatologically acceptable composition of any one of embodiments 40-41, wherein caffeine is present in an amount of about 0.25% to about 1% w/w of the composition.
43. The dermatologically acceptable composition of embodiment 42, wherein caffeine is present in an amount of about 0.01% to about 2% w/w of the composition.
44. The dermatologically acceptable composition of any one of embodiments 42-43, wherein caffeine is present in an amount of about 0.05% to about 1% w/w of the composition.
45. The dermatologically acceptable composition of any one of embodiments 1-44, wherein the phytochemical comprises farnesol.
46. The dermatologically acceptable composition of embodiment 45, wherein the farnesol is present in an amount of about 0.00001% to about 5% w/w of the composition.
47. The dermatologically acceptable composition of any one of embodiments 45-46, wherein the farnesol is present in an amount of about 0.00001% to about 2% w/w of the composition.
48. The dermatologically acceptable composition of any one of embodiments 45-47, wherein the farnesol is present in the composition as a component of an extract or oil from a plant tissue from a plant genus selected from the group consisting of: Cymbopogon, Prunus, Rosa, and a combination thereof.
49. The dermatologically acceptable composition of any one of embodiments 45-48, wherein the farnesol is present in the composition as a component of an extract or oil from a plant selected from the group consisting of: from a plant selected from the group consisting of: Vachellia farnesiana, Matricaria chamomilla; Abelmoschus moschatus; Myrocarpus fastigiatus; Oxystigma buccholtzii Harms; Cananga odorata; Acacia farnesiana; Myroxylon balsamum var. pereirae; Polianthes tuberosa; Pimpinella anisum; Aremisia campestris, Cyclamen Persicum, Myroxylon balsamum, Cymbopogon nardus, Cymbopogon winterianus, Cymbopogon martini, Cymbopogon schoenanthus, Cymbopogon Flexuosus Oi, Cymbopogon citratus, Prunus armeniaca, Rosa damascene, Rosa damascene, and a combination thereof.
50. The dermatologically acceptable composition of any one of embodiments 45-49, wherein the farnesol is present in the composition as a component of a lemon grass extract.
51. The dermatologically acceptable composition of embodiment 50, wherein the lemon grass extract is present in an amount of about 0.05% to about 2% w/w of the composition.
52. The dermatologically acceptable composition of any one of embodiments 6-7, wherein the composition further comprises an organic acid.
53. The dermatologically acceptable composition of any one of embodiments 1-4 and 8-52, wherein the organic acid is present in an amount of about 0.01% to about 5% w/w of the composition.
54. The dermatologically acceptable composition of any one of embodiments 1-4 and 8-53, wherein the organic acid is present in an amount of about 0.1% to about 3% w/w of the composition.
55. The dermatologically acceptable composition of any one of embodiments 1-4 and 8-54, wherein the organic acid is selected from the group consisting of: citric acid, malic acid, oxalic acid, manolic acid, malic acid, acetic acid, pyruvic acid, oxalic acid, glutaric acid, fumaric acid, formic acid, lactic acid, succinic acid, α-ketoglutaric acid, and a combination thereof.
56. The dermatologically acceptable composition of any one of embodiments 1-4 and 8-55, wherein the organic acid is present in the composition as a component of an extract from a citrus fruit.
57. The dermatologically acceptable composition of any one of embodiments 1-4 and 8-56, wherein the organic acid is present in the composition as a component of an extract from a citrus fruit selected from the group consisting of: amanatsu (Citrus natsudaidai), balady citron (Citrus medica), bergamot orange (Citrus bergamia), bitter orange (Citrus x aurantium), blood orange (Citrus x sinensis), Buddha's hand (Citrus medica var. sarcodactylis), calamondin (Citrus mitis), Cam sành (Citrus reticulata x maxima), citron (Citrus medica), clementine (Citrus reticulate), Corsican citron (Citrus medica), desert lime (Citrus glauca), etrog (Citrus medica), finger lime (Citrus australasica), Florentine citron (Citrus x limonimedica), grapefruit (Citrus x paradise), greek citron (Citrus medica), hyaganatsu (Citrus tamurana), Anadomikan (Citrus x iyo), kabosu (Citrus sphaerocarpa), kaffir lime (Citrus hystrix), key lime (Citrus aurantiifolia), kinnow (Citrus nobilis x Citrus deliciosa), kiyomi (Citrus unshiu x Citrus sinensis), kumquat (Citrus japonica), lemon (Citrus limon), sweet lime (Citrus limetta), mandarin orange (Citrus reticulata), mangshanyegan (Citrus mangshanensis), meyer lemon (Citrus x meyeri), Moroccan citron (Citrus medica), chinotto (Citrus myrtifolia), orange (Citrus x sinensis), oroblanco (Citrus grandis x C. Paradisi/Citrus maxima/Citrus grandis), a papeda, Persian lime (Citrus x latifolia), pomelo (Citrus maxima or Citrus grandis), ponderosa lemon (Citrus maxima x medica), Rangpur (Citrus x limonia), round lime (Citrus australis), satsuma (Citrus unshiu), shangjuan (Citrus ichangensis x C. maxima), shonan gold (Citrus flaviculpus hort. ex Tanaka (Ōgonkan) x Citrus unshiu), sudachi (Citrus sudachi), Taiwan tangerine (Citrus x depressa), tangelo (C. reticulata x C. maxima or x C. paradise), tangerine (Citrus tangerine), tangor (C. reticulata x C. sinensis), ugli fruit (Citrus reticulata x Citrus paradise), yuzu (Citrus ichangensis x C. reticulate), and a combination thereof.
58. The dermatologically acceptable composition of embodiment 57, wherein the extract from a citrus fruit is citrus limon fruit extract.
59. The dermatologically acceptable composition of any one of embodiments 56-58, wherein the extract from a citrus fruit is present in an amount of about 0.01% to about 5% w/w of the composition.
60. The dermatologically acceptable composition of any one of embodiments 56-59, wherein the extract from a citrus fruit is present in an amount of about 0.01% to about 1% w/w of the composition.
61. The dermatologically acceptable composition of any one of embodiments 6-7, wherein the composition further comprises an antimicrobial peptide.
62. The dermatologically acceptable composition of any one of embodiments 1-4 and 8-61, wherein the antimicrobial peptide is selected from the group consisting of: lugdunin, human β-defensin-1 (hBD1), hBD2, hBD3, CAP18, a hepcidin, cathelicidin peptide LL-37, dermcidin (DCD-1), adrenomedullin, elafin (SKALP), and a combination thereof.
63. The dermatologically acceptable composition of any one of embodiments 1-4 and 8-62, wherein the antimicrobial peptide is lugdunin.
64. The dermatologically acceptable composition of embodiment 63, wherein the lugdunin is present in an amount of about 1.5 to about 20.5 pg/mL of the composition.
65. The dermatologically acceptable composition of any one of embodiments 1-3 and 54-64, wherein the dermatologically acceptable composition comprises:
   a yeast component;
   caffeine;
   farnesol;
   one or more organic acids; and
   lugdunin.
66. The dermatologically acceptable composition of any one of embodiments 1-65, wherein the dermatologically acceptable carrier is selected from the group consisting of: arrowroot powder, ozokerite, carnauba wax, candelilla wax, beeswax, tapioca starch, cornstarch, propanediol, water, glycerin, sodium stearate, diatomaceous earth, polyglyceryl-6 caprylate, polyglyceryl-4 caprate, ethylhexylglycerin, triethyl citrate, Aloe barbadensis leaf juice, leuconostoc/radish root ferment filtrate, jojoba esters, stearyl alcohol, cellulose, silica, hydrated silica, titanium dioxide, and a combination thereof.
67. The dermatologically acceptable composition of any one of embodiments 1-66, wherein the dermatologically acceptable carrier comprises one or more of: a humectant, an emollient, a solubilizer and/or emulsifier, a preservative, and a matrix agent.
68. The dermatologically acceptable composition of embodiment 67, wherein the humectant comprises one or more of: erythritol, pentylene glycol, propanediol, sodium pyrrolidone carboxylic acid (PCA), sodium hyaluronate, betaine, glycerin, propylene glycol, a polyethylene glycol, a sugar, hexylene glycol, butylene glycol, aloe vera gel, an alpha hydroxy acid such as lactic acid, glyceryl triacetate, lithium chloride, sorbitol, xylitol, maltitol, hyaluronic acid, allantoin, urea, tremella extract, dicyanamide, sodium lactate, and sodium L-pyroglutamate urea, and pyrrolidone carboxylic acid.
69. The dermatologically acceptable composition of embodiment 68, wherein the humectant comprises glycerin.
70. The dermatologically acceptable composition of embodiment 67-69, wherein the humectant is present in an amount of about 15% to about 25% w/w of the composition.
71. The dermatologically acceptable composition of any one of embodiments 67-70, wherein the emollient comprises one or more of: elastin, cetyl alcohol, a silicone, a coconut alkane, coco-caprylate/caprate, diheptyl succinate, capryloyl glycerin/sebacic acid copolymer, triethyl citrate, caprylic/capric triglyceride, a butter (e.g., Butyrospermum parkii butter (shea butter) or Theobroma cacao (cocoa) seed butter), collagen, colloidal oatmeal, elastin, glyceryl stearate, isopropyl palmitate, shea butter, coconut oil (Cocos nucifera oil), and stearic acid.
72. The dermatologically acceptable composition of embodiment 71, wherein the emollient comprises stearic acid.
73. The dermatologically acceptable composition of any one of embodiments 67-72, wherein the emollient is present in an amount of about 1% to about 10% w/w of the composition.
74. The dermatologically acceptable composition of any one of embodiments 67-73, wherein the emollient is present in an amount of about 4% to about 8% w/w of the composition.
75. The dermatologically acceptable composition of any one of embodiments 67-74, wherein the solubilizer and/or emulsifier comprises one or more of: polyglyceryl-6 caprylate and polyglyceryl-4 caprate.
76. The dermatologically acceptable composition of any one of embodiments 67-75, wherein the solubilizer and/or emulsifier comprises one or more of: polyglyceryl-6 caprylate and polyglyceryl-4 caprate.
77. The dermatologically acceptable composition of embodiment 67-76, wherein the solubilizer and/or emulsifier is present in an amount of about 1% to about 5% w/w of the composition.
78. The dermatologically acceptable composition of any one of embodiments 67-77, wherein the matrix agent comprises one or more of: corn starch, sodium bicarbonate (baking soda), magnesium hydroxide, sodium hydroxide, arrowroot powder, a wax (e.g., ozokerite (earth wax), carnauba wax, candelilla wax, beeswax, microcrystalline wax, and Oryza sativa (rice) bran wax), tapioca starch, cornstarch, propanediol, water, sodium stearate, diatomaceous earth, cellulose, silica, hydrated silica, and titanium dioxide
79. The dermatologically acceptable composition of any one of embodiments 67-78, wherein the matrix agent is present in an amount of about 45% to about 65% w/w of the composition.
80. The dermatologically acceptable composition of any one of embodiments 67-79, wherein the matrix agent is present in an amount of about 50% to about 60% w/w of the composition.
81. The dermatologically acceptable composition of any one of embodiments 67-80, wherein the matrix agent comprises propanediol, water, sodium hydroxide, or a combination thereof.
82. The dermatologically acceptable composition of any one of embodiments 67-81, wherein the propanediol is present in an amount of about 30% to about 40% w/w of the composition.
83. The dermatologically acceptable composition of any one of embodiments 67-82, wherein the propanediol is present in an amount of about 10% to about 20% w/w of the composition.
84. The dermatologically acceptable composition of any one of embodiments 67-83, wherein the preservative is selected from the group consisting of: leuconostoc/radish root ferment filtrate, a benzoate, a benzoic acid, a propionate, propionic acid, a sorbate, sorbic acid, a salicylic acid, a salicylate, hexa-2,4-dienoic acid, a hexa-2,4-dienoate, formic acid, 3-acetyl-6-methylpyran-2,4-(3H)-dione, 3,3′-dibromo-4,4′-hexamethylenedioxydibenzamidine and its salts, thiomersal, phenylmercuric salts, undec-10-enoic acid and its salts, 1,3-bis (2-ethylhexyl) hexahydro-5-methyl-5-pyrimidine, 5-bromo-5-nitro-1,3-dioxane, bronopol, 2,4-dichlorobenzyl alcohol, 1-(4-chlorophenyl)-3-(3,4-dichlorophenyl) urea, chlorocresol, chloroxylenol, 5-chloro-2-(2,4-dichlorophenoxy) phenol, N,N″-methylenebis[N′-[3-(hydroxymethyl)-2,5-dioxoimidazolidin-4-yl]urea], polyaminopropyl biguanide, methenamine, quaternium-15, climbazole, DMDM hydantoin, benzyl alcohol, 1-Hydroxy-4-methyl-6-(2,4,4-trimethylpentyl)-2-pyridon, piroctone olamine, bromochlorophene, o-cymen-5-ol, chlorophene, chloroacetaminde, methylchloroisothiazolinone, methylisothiazolinone, phenoxyisopropanol, chlorhexidine, chlorhexidine diacetate, chlorhexidine digluconate, chlorhexidine dihydrochloride, dimethyl oxazolidine, behentrimonium chloride, cetrimonium bromide, cetrimonium chloride, laurtrimonium bromide, laurtrimonium chloride, steartrimonium bromide, steartrimonium chloride, diazolidinyl urea, hexamidine, hexamidine diisethionate, hexamidine paraben, glutaral, 7-ethylbicyclooxazolidine, chlorphenesin, sodium hydroxymethylglycinate, silver chloride, benzethonium chloride, benzalkonium chloride, benza-lkonium bromide, benzalkonium saccharinate, benzylhemiformal, iodopropynyl butylcarbamate, biphenyl-2-ol and its salts, pyrithionine zinc, an erythorbate, a nitrite, ethylenediaminetetraacetic acid (EDTA), butylated hydroxyanisole (BHA), butylated hydroxytoluene (BHT), capryllic acid, dilauryl thiodipropionate, erythorbic acid, gum guaiac, methylparaben, a sulfite, a bisulfite, a metabisulfite, propyl gallatepy, propylparaben, stannous chloride, sulfur dioxide, thiodipropionic acid, an isothiazoline, a paraben, phenoxyethanol, ethylhexylglycerin, a glycol, caprylhydroxamic acid, caprylyl glycol, glyceryl capylate, sodium benzoate, potassium sorbate, 1,2-hexanediol, propanediol, and a tocopherol.
85. The dermatologically acceptable composition of any one of embodiments 1-84, wherein the dermatologically acceptable carrier comprises one or more of: a humectant, an emollient, and a matrix agent.
86. The dermatologically acceptable composition of any one of embodiments 1-85, wherein the dermatologically acceptable carrier comprises one or more of: a humectant, an emollient, and a matrix agent.
87. The dermatologically acceptable composition of any one of embodiments 1-86, wherein the dermatologically acceptable carrier comprises propanediol, water, glycerin, stearic acid, and sodium hydroxide.
88. The dermatologically acceptable composition of any one of embodiments 1-87, wherein the dermatologically acceptable carrier comprises propanediol, water, glycerin, stearic acid, sodium hydroxide, silica, and hydrated silica.
89. The dermatologically acceptable composition of embodiment 87 or 88, wherein the dermatologically acceptable carrier further comprises one or more of: diatomaceous earth, Maranta arundinacea (arrowroot) root powder, polyglyceryl-6 caprylate, polyglyceryl-4 caprate, leuconostoc/radish root ferment filtrate, Aloe barbadensis leaf juice, jojoba esters, cellulose, ethylhexylglycerin, triethyl citrate, stearyl alcohol, and titanium dioxide.
90. The dermatologically acceptable composition of any one of embodiments 1-80, wherein the composition further comprises an antiperspirant.
91. The dermatologically acceptable composition of embodiment 81, wherein the antiperspirant comprises one or more of: aluminum chloride, aluminum chlorohydrate, aluminum chlorohydrex polyethylene glycol complex, aluminum chlorohydrex propylene glycol complex, aluminum dichlorohydrate, aluminum dichlorohydrex polyethylene glycol complex, aluminum dichlorohydrex propylene glycol complex, aluminum sesquichlorohydrate, aluminum sesquichlorohydrex polyethylene glycol complex, aluminum sesquichlorohydrex propylene glycol complex, aluminum sulfate buffered with sodium aluminum lactate, aluminum zirconium octachlorohydrate, aluminum zirconium octachlorohydrex glycine complex, aluminum zirconium pentachlorohydrate, aluminum zirconium pentachlorohydrex glycine complex, aluminum zirconium tetrachlorohydrate, aluminum zirconium tetrachlorohydrex glycine complex, aluminum zirconium trichlorohydrate, and aluminum zirconium trichlorohydrex glycine complex.
92. The dermatologically acceptable composition of any one of embodiments 1-92, wherein the composition is configured for topical application.
93. The dermatologically acceptable composition of any one of embodiments 1-93, wherein the composition is in the form of a solid, a semi-solid, a lotion, an emulsion, or a liquid.
94. The dermatologically acceptable composition of any one of embodiments 1-94, wherein the composition is in the form of liquid and configured as a spray or a roll-on.
95. A method for reducing body odor comprising applying a dermatologically acceptable composition of any one of embodiments 1-94 to a subject.
96. A method for reducing the production of one or more malodorous compounds by bacteria in sweat comprising applying a dermatologically acceptable composition of any one of embodiments 1-94 to a subject.
97. The method of embodiment 96, wherein the one or more malodorous compounds are selected from the group consisting of: a thioalcohol, acetic acid, isovaleric acid, 3-methyl-2-hexenoic acid, propionic acid, and 3-hydroxy-3-methylhexanoic acid.
98. The method of embodiment 96 or 97, wherein the bacteria are from a genus selected from the group consisting of: Corynebacterium, Micrococcus, Staphylococcus, Propionibacterium, Brevibacterium, Cutibacterium, and a combination thereof.
99. The method of embodiment 98, wherein the bacteria are from a species selected from the group consisting of: S. hominis, S. aureus, S. epidermidis, Cutibacterium avidum, and a combination thereof.
100. The method of any one of embodiments 95-99, wherein the composition is applied to the subject on an area selected from the group consisting of: an underarm, the face, a heel of a foot, a hairline, an inner thigh, an area behind a knee, a sole of a foot, a hand, the groin, genitalia, perineum and a combination thereof.
101. The method of any one of embodiments 95-100, wherein the dermatologically acceptable composition is applied daily.
102. The method of any one of embodiments 95-101, wherein the dermatologically acceptable composition is applied once every two days or once every three days.

It is to be understood that while the invention has been described in conjunction with the detailed description thereof, the foregoing description is intended to illustrate and not limit the scope of the invention which is defined by the scope of the appended claims. Other aspects, advantages, and modification are within the scope of the following claims.

1. A dermatologically acceptable composition comprising:
A) a yeast component;
B) a phytochemical; an organic acid; or a combination thereof; and
C) a dermatologically acceptable carrier.

2. The dermatologically acceptable composition of claim 1, wherein the yeast component is a yeast extract a yeast cell wall, or a combination thereof.

3. (canceled)

4. The dermatologically acceptable composition of claim 1, wherein the yeast component is present in an amount of about 0.5% to about 20% w/w of the composition.

5. (canceled)

6. (canceled)

7. The dermatologically acceptable composition of claim 1, wherein the yeast extract comprises: a Saccharomyces extract, a Candida extract, a Debaryomyces extract, a Kloeckera extract, a Kluyveromyces extract, a Geotrichum extract, a Pichia extract, a Wickerhamomyces extract, or a combination thereof.

8.-12. (canceled)

13. The dermatologically acceptable composition of claim 1, wherein the phytochemical is present in an amount of about 0.1% to about 10% w/w of the composition.

14. (canceled)

15. (canceled)

16. The dermatologically acceptable composition of claim 1, wherein the phytochemical is selected from the group consisting of: farnesol, caffeine, caffeic acid, chlorogenic acid, a derivative of chlorogenic acid, tannic acid, trigonolline, protocatechuic acid, and a combination thereof.

17. (canceled)

18. The dermatologically acceptable composition of claim 1, wherein the phytochemical comprises caffeine, farnesol, or a combination thereof.

19.-22. (canceled)

23. The dermatologically acceptable composition of any one of claim 18, wherein the farnesol is present in the composition as a component of an extract or oil from a plant selected from the group consisting of: from a plant selected from the group consisting of: Vachellia farnesiana, Matricaria chamomilla; Abelmoschus moschatus; Myrocarpus fastigiatus; Oxystigma buccholtzii Harms; Cananga odorata; Acacia farnesiana; Myroxylon balsamum var. pereirae; Polianthes tuberosa; Pimpinella anisum; Aremisia campestris, Cyclamen Persicum, Myroxylon balsamum, Cymbopogon nardus, Cymbopogon winterianus, Cymbopogon martini, Cymbopogon schoenanthus, Cymbopogon Flexuosus Oi, Cymbopogon citratus, Prunus armeniaca, Rosa damascene, Rosa damascene, and a combination thereof.

24. (canceled)

25. (canceled)

26. The dermatologically acceptable composition of claim 1, wherein the organic acid is selected from the group consisting of: citric acid, malic acid, oxalic acid, manolic acid, malic acid, acetic acid, pyruvic acid, oxalic acid, glutaric acid, fumaric acid, formic acid, lactic acid, succinic acid, α-ketoglutaric acid, and a combination thereof.

27. The dermatologically acceptable composition of claim 1, wherein the organic acid is present in the composition as a component of an extract from a citrus fruit.

28. (canceled)

29. (canceled)

30. The dermatologically acceptable composition of claim 27, wherein the extract from a citrus fruit is present in an amount of about 0.01% to about 5% w/w of the composition.

31.-33. (canceled)

34. The dermatologically acceptable composition of claim 1, wherein the dermatologically acceptable carrier is selected from the group consisting of: arrowroot powder, ozokerite, carnauba wax, candelilla wax, beeswax, tapioca starch, cornstarch, propanediol, water, glycerin, sodium stearate, diatomaceous earth, polyglyceryl-6 caprylate, polyglyceryl-4 caprate, ethylhexylglycerin, triethyl citrate, Aloe barbadensis leaf juice, leuconostoc/radish root ferment filtrate, jojoba esters, stearyl alcohol, cellulose, silica, hydrated silica, titanium dioxide, and a combination thereof.

35. The dermatologically acceptable composition of claim 1 wherein the dermatologically acceptable carrier comprises one or more of: a humectant, an emollient, a solubilizer and/or emulsifier, a preservative, and a matrix agent.

36. The dermatologically acceptable composition of claim 35, wherein the humectant comprises one or more of: erythritol, pentylene glycol, propanediol, sodium pyrrolidone carboxylic acid (PCA), sodium hyaluronate, betaine, glycerin, propylene glycol, a polyethylene glycol, a sugar, hexylene glycol, butylene glycol, aloe vera gel, an alpha hydroxy acid such as lactic acid, glyceryl triacetate, lithium chloride, sorbitol, xylitol, maltitol, hyaluronic acid, allantoin, urea, tremella extract, dicyanamide, sodium lactate, and sodium L-pyroglutamate urea, and pyrrolidone carboxylic acid.

37. The dermatologically acceptable composition of claim 35, wherein the emollient comprises one or more of: elastin, cetyl alcohol, a silicone, a coconut alkane, coco-caprylate/caprate, diheptyl succinate, capryloyl glycerin/sebacic acid copolymer, triethyl citrate, caprylic/capric triglyceride, a butter (e.g., Butyrospermum parkii butter (shea butter) or Theobroma cacao (cocoa) seed butter), collagen, colloidal oatmeal, elastin, glyceryl stearate, isopropyl palmitate, shea butter, coconut oil (Cocos nucifera oil), and stearic acid.

38. The dermatologically acceptable composition of claim 35, wherein the solubilizer and/or emulsifier comprises one or more of: polyglyceryl-6 caprylate and polyglyceryl-4 caprate.

39. The dermatologically acceptable composition of claim 35, wherein the matrix agent comprises one or more of: corn starch, sodium bicarbonate (baking soda), magnesium hydroxide, sodium hydroxide, arrowroot powder, a wax (e.g., ozokerite (earth wax), carnauba wax, candelilla wax, beeswax, microcrystalline wax, and Oryza sativa (rice) bran wax), tapioca starch, cornstarch, propanediol, water, sodium stearate, diatomaceous earth, cellulose, silica, hydrated silica, and titanium dioxide

40.-44. (canceled)

45. The dermatologically acceptable composition of claim 1, wherein the composition further comprises an antiperspirant.

46.-48. (canceled)

49. A method for reducing body odor comprising applying a dermatologically acceptable composition of claim 1 to a subject.

50. A method for reducing the production of one or more malodorous compounds by bacteria in sweat comprising applying a dermatologically acceptable composition of claim 1 to a subject.

51.-56. (canceled)
